(19) United States
(12) Patent Application Publication
(10) Pub. No.: US 2023/0120068 A1
(43) Pub. Date: Apr. 20, 2023

(54) CLEANROOM-FREE INTEGRATED TRIMODAL SILICON TRANSDUCER FOR GENETIC DETECTION OF PATHOGENS

(71) Applicants: IMPERIAL COLLEGE INNOVATIONS LIMITED, London (GB); MOREDUN RESEARCH INSTITUTE, Edinburgh, Scotland (GB)

(72) Inventors: Estefania NUNEZ-BAJO, London (GB); Firat GUDER, London (GB); Karen STEVENSON, Edinburgh, Scotland (GB)

(21) Appl. No.: 17/906,220

(22) PCT Filed: Mar. 12, 2021
(86) PCT No.: PCT/EP2021/056332

§ 371 Date: Sep. 13, 2022

(30) Foreign Application Priority Data
Mar. 13, 2020 (GB) 2003649.7

(51) Int. Cl. G01N 27/327; C12Q 1/70; B01L 3/00; B01L 7/00; C25D 5/54; C25D 7/00

(52) U.S. Cl. CPC G01N 27/3277; C12Q 1/701; B01L 3/502707; B01L 7/52; C25D 5/54; C25D 7/00; G01N 27/3276; C12Q 1/6844

(57) ABSTRACT

A device for detecting a substance in a solution, wherein the device comprises: a substrate comprising silicon; a first electrode for use as a working electrode in an electrochemical cell and coupled to the substrate; and Ohmic contacts coupled to the substrate and configured to pass a current through the substrate when connected to a power source.

The present application relates to a device and a system for detecting a substance in a solution, methods of fabricating and methods of using the same. Applications of the device and system include the detection of nucleic acids through electrochemistry.

Despite the advancement of diagnostic technologies targeting nucleic acids (NA), there are no portable, disposable and integrated solutions for the testing of infectious diseases at the point of care (POC). Usually, the amount of pathogenic DNA in the sample is not enough high for the direct detection and it is necessary to recur to NA amplification methods, increasing the complexity of the analysis. DNA amplification strategies were developed for sensitive detection of DNA, specially, polymerase chain reaction (PCR). Conventional PCR requires using voluminous, high energy-consuming and expensive thermocyclers. Therefore, many efforts have been employed in the design of miniaturized thermocyclers and in the development of isothermal DNA amplification strategies which do not require thermoregulated equipment. However, some isothermal DNA amplification techniques have complicated reaction mechanisms and experimental designs.

To date, several integrated NA-based analytical systems have been commercialized such as GeneXpert MTB/RIF. Despite their wide use in clinical/central laboratories, their application for on-site pathogen monitoring on a routine basis is still limited due to the large footprint and high instrument cost. The miniaturization of NA analytical platforms has many advantages over the conventional bench-top counterparts. These include low sample/reagent consumption (volume of micro- down to picoliter) as well as short assay time (minutes rather than days). Most importantly, they permit the integration of a number of functions including sample preparation, target amplification, and product detection, thus enabling a fully automated operation that can be used by untrained individuals. There are two ways to perform NA amplification. One is time-domain, in which a reservoir or chamber (pool) allows temperature changing with PCR cycling. The other way is space-domain, which executes thermal cycles by moving the reaction mixture between different temperatures zones. The design of the miniaturized NA amplification device depends mainly on the type (isothermal or PCR) and the domain (time or space domain) of the NA amplification.

Fluorescence detection (FD) is the traditional way to detect DNA in routine analysis. However, it requires fluorescent labelling and an expensive optical detection system that is difficult to miniaturize and is only adopted in centralised laboratories. Besides, FD has the disadvantage of high background interference. On the other hand, electrochemical detection (EC) is easily miniaturizable and EC-DNA analysis displays rapid, sensitive, simple, non-toxic, low-cost and effective merits compared to the shortcomings of FD. The application of EC technology in PCR amplification is known.

Micro-devices that integrate time-domain PCR amplification with EC detection (PCR-EC) are scarce in the literature (Fang, X. et al. Real-time monitoring of strand-displacement DNA amplification by a contactless electrochemical microsystem using interdigitated electrodes. Lab Chip 12, 3190-3196 (2012); Yeung, S.-W., Lee, T. M.-H., Cai, H. & Hsing, I.-M. A DNA biochip for on-the-spot multiplexed pathogen identification. Nucleic Acids Res. 34, e118 (2006); Petralia, S. et al. A miniaturized silicon-based device for nucleic acids electrochemical detection. Sens. Bio-Sensing Res. 6, 90-94 (2015); Yeung, S. S. W., Lee, T. M. H. & Hsing, I.-M. Electrochemistry-Based Real-Time PCR on a Microchip. Anal. Chem. 80, 363-368 (2008). Lee, T. M.-H., Carles, M. C. & Hsing, I.-M. Microfabricated PCR-electrochemical device for simultaneous DNA amplification and detection. Lab Chip 100-10 (2003)). The fabrication methods of the reported devices are time-consuming, expensive or require special materials, equipment or cleanroom facilities. In addition, these devices use metal deposited on silicon as heater and resistance temperature detector (RTD) without exploiting the thermoelectric properties of the silicon as thermistor. Thermistors differ from RTDs in that the material used in a thermistor is generally a ceramic or polymer, while RTDs use pure metals. The temperature response is also different; RTDs are useful over larger temperature ranges, while thermistors typically achieve a greater precision within a limited temperature range, typically −90° C. to 130° C. Since RTDs are active sensors, they require external excitation to produce a measurable voltage drop that can be translated into resistance. Resistance values are generally very low, meaning lead-wire resistance can cause less accurate measurements. Because of this, RTDs often come in multiwire configurations and the measurement hardware must be carefully selected.

There is therefore a need for a device providing a low-cost, simple, structured, portable PCR-EC platform.

### SUMMARY OF THE INVENTION

Described herein is a device and a system for detecting a substance in a solution, methods of fabricating and methods of using the same.

According to a first aspect, the invention provides device for detecting a substance in a solution, wherein the device comprises:
a substrate comprising silicon;
a first electrode for use as a working electrode in an electrochemical cell and coupled to the substrate; and
Ohmic contacts coupled to the substrate and configured to pass a current through the substrate when connected to a power source.

According to a second aspect, the invention provides a system for detecting a substance in a solution, the system comprising:
a device according to the first aspect of the invention; and one or more of:
   a potentiostat configured to control an electrochemical potential of the first electrode;
   a power source configured to pass a current through the substrate via the Ohmic contacts; and
   a control unit connected to the Ohmic contacts and configured to calculate a resistance of the substrate.

According to a third aspect, the invention provides a method of fabricating a device according to the first aspect of the invention comprising
electroplating the first electrode to the substrate, thereby coupling the first electrode to the substrate, and
electroplating the Ohmic contacts to the substrate thereby coupling the Ohmic contacts to the substrate.

According to a fourth aspect, the invention provides a method of detecting a substance in a solution using a device according to the first aspect of the invention, the method comprising
placing the solution in contact with the first electrode,
passing a current through the substrate, and
detecting a substance in the solution.

According to a fifth aspect, the invention provides a method of diagnosis using a device according to the first aspect of the invention, the method comprising
placing a sample in contact with the first electrode,
passing a current through the substrate, and
detecting a substance in the sample.

Reference is made to a number of Figures as follows:

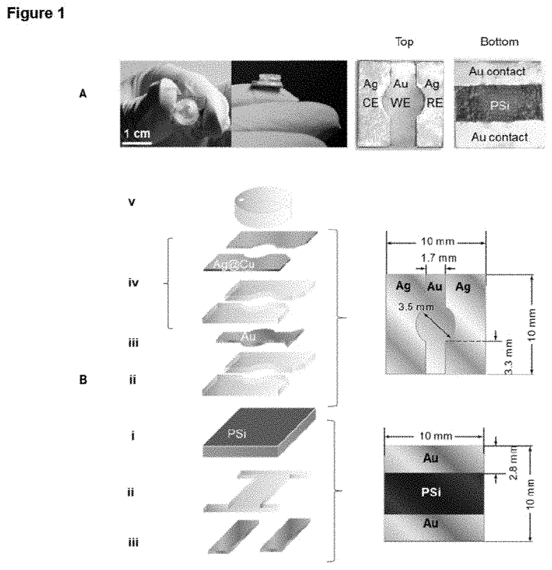

FIG. 1. A) Pictures of the final rt-PCR-EC device. B) Schematic representation by layers and top and bottom views of the device.

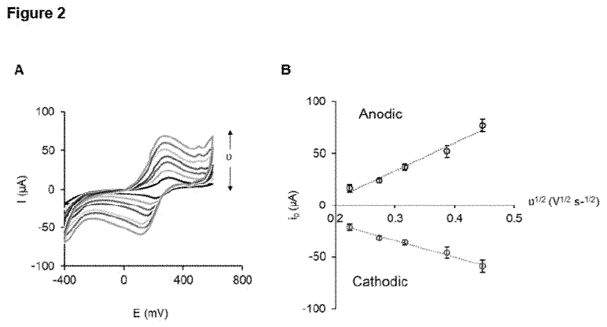

FIG. 2. A) CVs in 2 mM K₄[Fe(CN)₆] solution (0.1 M KCl) sweeping the potential from −600 to +700 mV at 10, 50, 75, 100, 150 and 200 mV s⁻¹. B) Peak current intensity vs scan rate plots from CVs recorded using 5 devices.

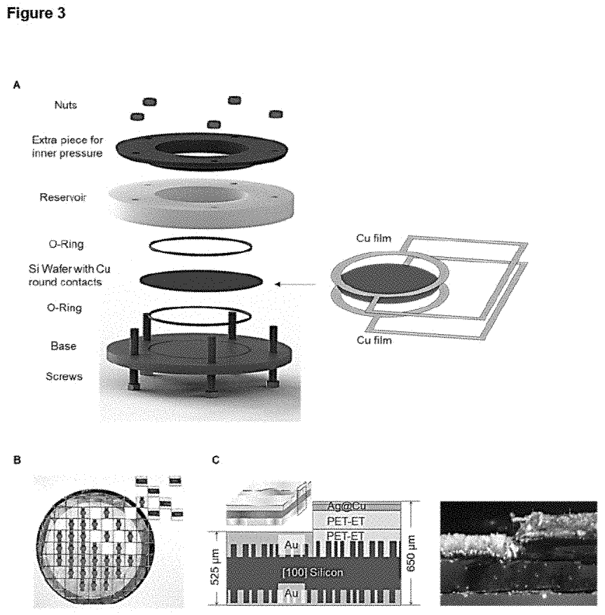

FIG. 3. A) 3D-printed PLA holder developed for wafer-scale electroplating. B) Picture of the devices obtained after wafer-scale manufacturing and laser cutting. C) Schematic illustration (right) and optical micrograph (left) of the cross section of the final device.

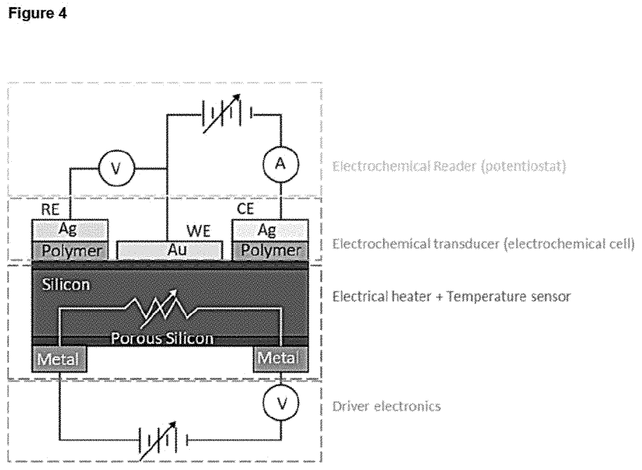

FIG. 4. Schematic representation of the setup of the device

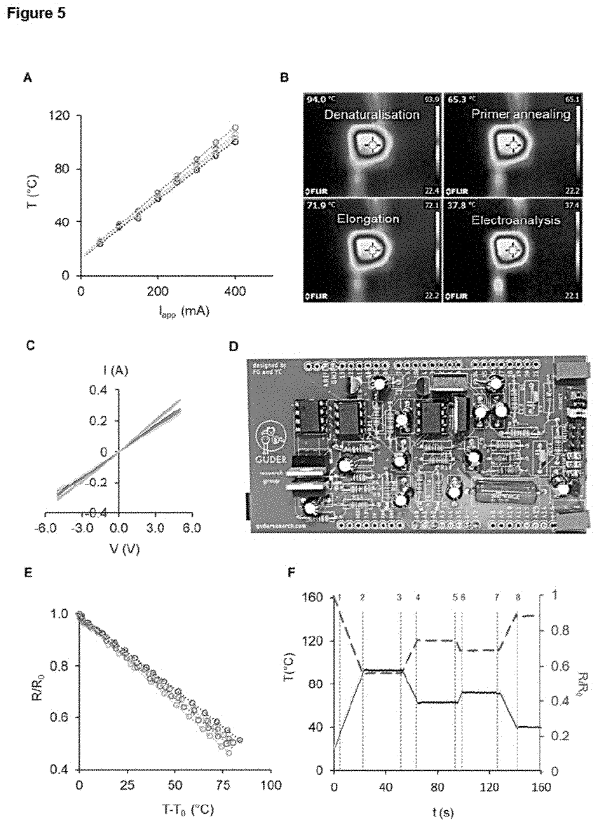

FIG. 5. Thermoelectric characterization of the Au-PSi-Au diode. A) Temperature-current curves and B) related IR images recorded during the experiment. C) Current-voltage curves of the diode. D) Custom circuit board used for heating control and precise thermal sensing. E) Resistance-temperature curves plotted from resistance measurements using the custom board and software. F) Real (black line) and estimated (grey line adjacent to black line) thermal cycling temperature-time curves for one PCR cycle. Estimated temperature values were calculated from recorded resistance-time curve (upper dashed line).

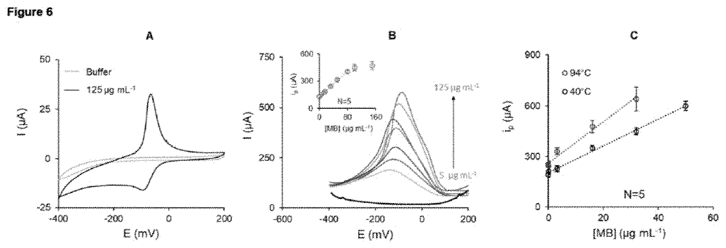

FIG. 6. A) CVs recorded in 125 μg mL⁻¹ MB solution in PBS. 100 mV s⁻¹. B) SWVs recorded in MB solution in PBS at room temperature and corresponding calibration curve (inset) at room temperature. C) Calibration plots. Peak current intensities were measured from SWVs recorded in 0, 0.005, 0.05, 0.5, 5, 15, 32 and 50 pg mL⁻¹ MB in PBS at 40° C. after 5 min at 40 and 90° C. Esw: 100 mV; Es: 5 mV; f:50 Hz.

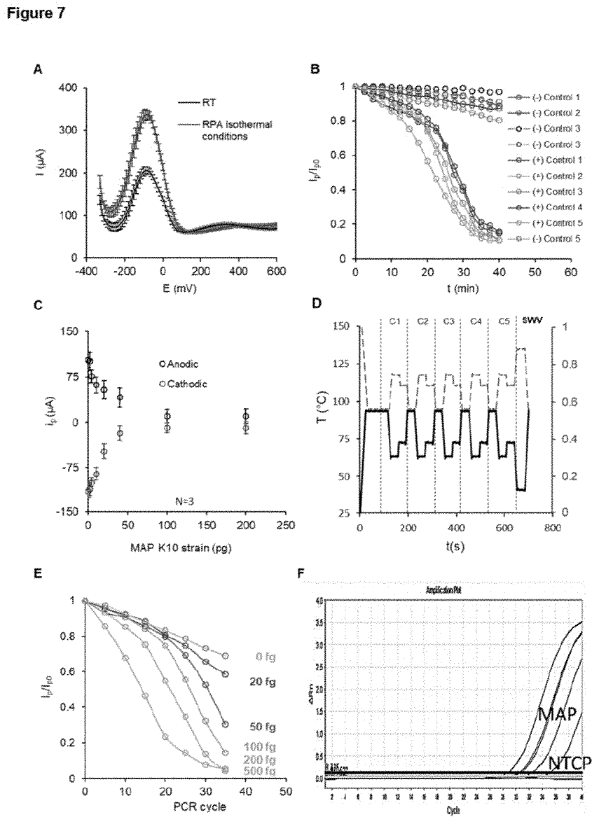

FIG. 7. A) Average signals from 18 SWVs recorded every 2.5 min in 10 μg mL⁻¹ MB RPA Mastermix. B) RT-RPA curves in positive and negative DNA control samples. C) Tritration curve at room temperature from SWVs recorded in 20 pg mL-1 MB MAP PCR mix solutions in a 2-200 pg range of K10 MAP strain. D) Thermal cycling curves of the first 5 cycles in MAP RT-PCR experiments. Vertical dashed lines delimitate each of the 5 PCR cycles performed before SVVV measurements. E) MAP RT-PCR curves in blank (0 fg) and MAP K10 solutions. Esw: 100 my; Es: 5 mV; f:50 Hz. F) Results obtained from the same MAP sample using IDVet commercial qPCR.

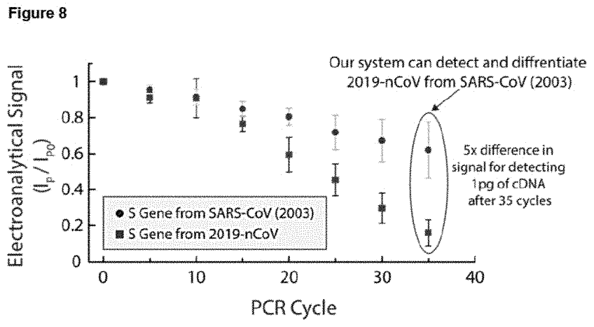

FIG. 8. Electroanalytical signal vs time curves during polymerase-chain amplification reactios (PCR) of 1 pg of SARS-CoV.2 (positive, 22712-22869 nucleotides of GenBank accession number MN908947) and 1 pg of SARS-CoV (negative, 17741-17984 nucleotides of GenBank accession number AY274119) cDNA fragments. Every PCR thermal cycle consisted of applying 94° C. for 30 s followed by 63° C. for 30 s and ending with 72° C. for 30 s. Resulting amplicons from the positive sample were 158 bp dsDNA. The electrochemical signal was recorded during the DNA amplification every 5 cycles sweeping the potential from +0.0 to −0.6 V and the peak current intensity (ip) measured from SWVs was normalized respect to that prior to amplification (p0). Frequency: 50 HZ; Pulse amplitude: 100 mV.

### DETAILED DESCRIPTION OF THE INVENTION

According to a first aspect, the invention provides a device for detecting a substance in a solution, wherein the device comprises:
a substrate comprising silicon;
a first electrode for use as a working electrode in an electrochemical cell and coupled to the substrate; and
Ohmic contacts coupled to the substrate and configured to pass a current through the substrate when connected to a power source.

The device may be a termed a microsensor. The device may for example be on a millimetre or tens of millimetre scale. The device may have a length, a width and a thickness. The thickness is typically around 0.6 mm. The length and width may be any suitable value. The length and width are typically the same. However, the length and width may alternatively be different. Suitable values for the length and/or width may be from around 5 mm to around 10 mm, such as 10 mm. Where the width is around 10 mm any suitable length may be used, including lengths greater than 10 mm. The minimum length and/or width may be 5 mm. For example, the microsensor may be around 10 mm×around 10 mm×around 0.6 mm in size. A 4-inch silicon wafer containing may contain around 37 microsensors each of around 10 mm×around 10 mm×around 0.6 mm in size. The 0.6 mm dimension is typically a thickness. The thickness is typically the thickness of the silicon wafer. In some embodiments, the device comprises a reaction chamber. These device measurements exclude the reaction chamber.

An advantage of the device of the invention is miniaturisation. Improved miniaturisation is enabled by the architecture of the device as claimed, which exploits the intrinsic properties of the substrate comprising silicon to provide three modes of operation in use:
i) an electrical thermal block,
ii) a temperature regulator with a negative temperature coefficient that can provide the precise temperature of the solution during use, for example a reaction, and
iii) an electrochemical sensor for detecting the substance in solution.

Existing portable devices are commonly fabricated by thin-film techniques in cleanrooms and use the silicon as substrate without exploiting its temperature dependent semiconducting properties. Exploiting the intrinsic properties of the substrate means that fewer additional components are required to support these three modes of operation, thus enabling improved miniaturisation. The miniaturization of analytical platforms has many advantages over conventional bench-top counterparts. These include lower sample/reagent consumption (volumes on a microliter to picolitre scale) as well as shorter assay times (minutes rather than days).

The substance in solution may be any substance suitable for detection by an electrochemical reporter. The substance in solution may be an electrochemical reporter.

The substance may be a biological molecule. The terms biological molecule and biomolecule are used interchangeably herein. The biological molecule may be a nucleic acid. The nucleic acid may be DNA, RNA or cDNA.

The biological molecule may be a protein, a peptide or a polypeptide. For example, the biological molecule may be a hormone or a neurotransmitter).

The biomolecule may be electroactive (e.g. dopamine). The biomolecule may be linked/conjugated to enzymes which produce or consume electroactive molecules (e.g. glucose oxidase, which catalyses the oxidation of glucose producing hydrogen peroxide, alkaline phosphatase, which catalyses the reaction of p-aminophenyl phosphate to p-aminophenol and horseradish peroxidase, which is re-oxidized by using electroactive mediators such as ferrocyanide). The biomolecule may be conjugated to electroactive species. The electroactive species may be organometallic complexes (e.g. ferrocene), organic molecules (e.g. methylene blue) or nanomaterials (e.g. quantum dots, gold nanoparticles, silver nanoparticles).

Nucleic acid detection can be based on electrostatic interactions with an electroactive reporter (e.g. [Ru(NH3)₆]Cl₃, K₃[Fe(CN)₆]); groove-binding reporters (e.g. Hoechst 33258); intercalation of the reporter using metalointercalators (e.g. bipyridyl and phenanthroline complexes of Ru, Co, Cu, Os and Fe), using organic intercalators (e.g. Ethidium Bromide, Anthraquinone); or a combination of electrostatic and intercalation interactions (e.g. Methylene blue).

The electrochemical reporter may be any suitable electrochemically active or electrically conductive material. The terms “electrochemical” and “electroactive” are used interchangeably herein. The skilled person will choose any suitable electrochemical reporter for the substance they intend to detect.

When the substance is a nucleic acid, the electrochemical reporter may be an intercalating agent. The intercalating agent may be selected from the group consisting of methylene blue, EthidiumBromine, Anthraquinone, M[(bpy)₂phen]²⁺, M[(bpy)2DPPZ]²⁺, M[(4,40-dimethyl-bpy)2DPPZ]²⁺ and M[(4,40-diamino-bpy)2DPPZ]²⁺ (with M=ROI), Os(ll), Cu(II), Fe(II); bpy=2,20-bipyridine; phen=phenanthroline; and DPPZ=dipyrido[3,2-a:20,30-c]phenazine), [Co(bpy)₃]³⁺ and [Co(phen)₃]³⁺. The intercalating agent may be methylene blue.

When the substance is a nucleic acid, the electrochemical reporter may be conjugated to a probe. The probe may be a nucleic acid complementary to a target sequence of nucleic acid. The probe may be an “eTaq” probe (Luo et al (2011) Electrochemistry Communications 13(7):742-745). Nucleic acid detection can be based on the use of probes or dNTPs conjugated to electroactive reporters.

The electrochemical reporter is in the solution. The substance interacts with the electrochemical reporter causing a change in its concentration in the solution. The change in concentration of the electrochemical reporter will produce an electrochemical signal. The electrochemical signal can be detected by the electrochemical cell.

For example, when the nucleic acid is DNA, the electrochemical signal is due to reporter molecules which have not interacted with the DNA. As more reporter molecules are intercalated in the DNA, the electronic transfer between the intercalated reporter and the electrode is reduced, so the electrochemical signal observed is due to those molecules of reporter that haven't interacted with DNA and are still free in solution. Therefore, the assay may be a signal-off assay where the increase in the concentration of the substance (in this case DNA) produces a decrease in the signal of the reporter (in this case methylene blue free in solution).

Detecting is therefore detecting by electrochemical means involving the electrochemical cell.

The device may therefore be termed a device for determining the concentration of a substance in a solution. “Determining” may mean “measuring” or “inferring”. The determination of a concentration may be indirect and require a calculation known to the person skilled in the art.

The substrate comprises silicon. Silicon has many advantages including its low-cost, easy microfabrication, small thermal expansion coefficient and a sufficiently high melting point. These properties also make silicon an ideal material for the construction of electrochemical platforms, including for amplification of nucleic acids.

The substrate may be around 100 μm to around 700 μm in thickness. The substrate may be around 500 μm to around 550 μm in thickness. The substrate may be around 525 μm in thickness. The substrate may be around 525±25 μm thickness.

The substrate may have a resistivity of around 0 to around 100 Ω·cm. The substrate may have a resistivity of around 50 ±50 Ω·cm. The substrate may have a resistivity of around 50 Ω·cm.

The silicon may be p-type Silicon. The p-type silicon may be Boron doped. The silicon may have <100> or <111> orientation. A <100> origination may improve the etching rate and anisotropy, for example in wet etching of bulk silicon. However, a <111> orientation slows the etching process, so a <111> orientation may be incorporated as an etch stop. The orientation does not matter when using a surface micromachining technology because the silicon serves only as a mechanical support. The silicon may have a concentration of dopant of around 1×10¹⁶ atoms per cubic centimetre to around 1×10¹⁷ atoms per cubic centimetre, for example around 1.50×10¹⁶ atoms per cubic centimetre The silicon may be termed “lightly-medium doped” silicon.

In use, the passage of an electric current through the silicon generates heat.

In use, the temperature of the silicon may be measured by detecting changes in resistance. The change in resistance may be a change in resistance of the porous silicon. The change in resistance may be detected using a resistance temperature detector.

The substrate may comprise bulk silicon. The substrate may comprise multiple types of silicon. The substrate may comprise bulk silicon and porous silicon. The substrate may comprise bulk silicon and silicon dioxide. The substrate may comprise bulk silicon, porous silicon and silicon dioxide. The substrate may comprise bulk silicon and optionally porous silicon and/or silicon dioxide. Typically, the types of silicon form layers in the substrate. The substrate may comprise one or more bulk silicon layer and optionally one or more porous silicon layers and/or one or more silicon dioxide layers. Typically, any porous silicon layers are on an outer surface of the substrate. Typically, any silicon dioxide layers are in the substrate interior. Typically, any silicon dioxide layers are not substantially on an outer surface of the substrate.

In some alternative embodiments, the substrate may comprise a semiconductor material other than silicon. The substrate can be any material able to act as thermistor (change in the resistance with the temperature) such as, metal oxides, single-crystal semiconductors and electroceramics. Semiconductor thermistors (Ge, GaAs, Si and SiC) may find particular use at relatively low temperatures of lower than 500° C. Germanium thermistors are more widely used than their silicon counterparts and are typically used for temperatures below 200° C. Silicon thermistors can be used at temperatures up to around 500° C. The thermistor itself may be made from a single crystal which has been doped to a level of 10¹⁶ - 10¹⁷ per cubic centimetre. Positive temperature coefficient (PTC) resistors are based on BaTiO₃ electroceramic materials.

The substrate can also be SOI wafer (silicon on insulator) where a layer of silicon oxide is in between two silicon layers which would insulate electrically the electrochemical cell, at the top of the device, from the heater/thermistor at the bottom.

Porous silicon (PSi) gains more importance in amperometric-based sensors because of the sensitivity enhancement due to the higher surface area of PSi compared to flat electrodes. The main difficulty is the low conductivity of PSi compared to metal electrodes. This can be overcome by coupling PSi with noble metals such as Pt, and Au, or a conductive polymer thin film to enhance its conductivity. Indeed, porous electrodes can increase biosensor sensitivity in comparison with flat surface electrodes.

The first electrode may be any suitable electrode for use as a working electrode in an electrochemical cell. The first electrode may be a porous electrode.

The working electrode may be referred to by the abbreviation “WE”. The effect of the working electrode is to enable function in use as an electrochemical cell. Compared to florescence-based devices, the device of the invention may be easier to use by untrained personnel; electrochemical detection is less susceptible to contamination than florescence-based detection.

The working electrode may be of any suitable size. The working electrode may have a longest dimension of about 1.5 mm to about 4 mm. The working electrode may have a longest dimension of about 3.5 mm to about 4 mm. The working electrode may have a longest dimension of about 4 mm. The longest dimension may be a diameter. The longest dimension may be a length.

The first electrode may comprise a noble metal or carbon. For example the first electrode may comprise gold, platinum, iridium, ruthenium, glassy carbon, carbon black, graphite or graphene. In a preferred embodiment, the first electrode comprises gold. The first electrode may comprise any suitable non-reactive material. The first electrode may be an indium tin oxide gold electrode. The first electrode may be porous silicon optionally comprising an immobilised enzyme. The first electrode may be a Polypyrrole (PPy) film. The first electrode may be gold nanostructured porous silicon. The first electrode may be a gold electrode.

The first electrode may be in electrical contact with the substrate.

An electrochemical cell may be known as an electrochemical transducer. The electrochemical cell may be connected to an electrochemical reader, or potentiostat, and may transmit a signal related to species capable of exchanging electrons with the working electrode (WE) or susceptible to redox reactions.

The device may further comprise a second electrode for use as a counter electrode in the electrochemical cell, wherein the second electrode is coupled to the silicon comprising substrate via a first insulating layer. The first insulating layer does not conduct electricity. It is therefore an electrical insulator. The first insulating layer may conduct heat.

The counter electrode may be referred to by the abbreviation “CE”. The counter electrode may be known as an auxiliary electrode, for example a first auxiliary electrode. The effect of the counter electrode is to improve the function of the electrochemical cell.

The counter electrode may be of any suitable size.. The counter electrode may have a longest dimension of about 3.5 mm to about 6mm. The counter electrode may have a longest dimension of about 6 mm. The longest dimension may be a length. The counter electrode may have a shortest dimension of about 1 mm to about 2 mm, for example 1.5 mm. The shortest dimension may be a width.

Without being bound by theory, the purpose of the counter electrode (CE) is to provide a pathway for current to flow in the electrochemical cell without passing significant current through the reference electrode. Any suitable material may be used. The skilled person will understand there are no specific material requirements for the electrode beyond it not adversely influencing reactions occurring at the working electrode (WE). If a reduction occurs at the WE, there must be an oxidation that takes place at the CE. Care should be taken that electrode products formed at the CE do not interfere with the WE reaction. The CE can be physically separated from the WE compartment, for example by using a fritted tube. The most commonly used material for the auxiliary electrode is platinum, due to its inertness and the speed with which most electrode reactions occur at its surface. Other, less expensive materials may also be used as auxiliary electrodes. Choices include carbon, copper, or stainless steel if corrosion is not an issue for a particular electrolyte solution or reaction.

The second electrode may comprise a metal or carbon. For example the first electrode may comprise gold, platinum, carbon, copper, or stainless steel. In a preferred embodiment, the second electrode comprises platinum. The second electrode may be a platinum electrode.

The insulating layer may be any suitable insulator. The insulating layer may be a thermoplastic material, a thermoset material or silicon dioxide. A silicon dioxide insulating layer may be formed through the generation of oxide on the silicon surface. Preferably, the insulating layer is a thermoplastic material or a thermoset material. For example, the insulating layer may be mPPE (modified polyphenylene ether), polyethylene terephthalate (PET), thermoplastic elastomer (TPE) or polyethylene (PE). Thermoplastic materials and a thermoset materials may be attached to the substrate by a thermal binding method. Thermoplastic and thermoset materials may be advantageous. because they can be repeatedly heated, softened, and formed into any shape when hot, due to their chain of molecules that separate when heat is applied. They are usually lower in cost, lighter in weight, easier to colour and have improved electrical properties.

The insulating layer may be of any suitable thickness to prevent conductivity between the electrodes causing a short circuit. For example, when PET or PE are the insulator material, the insulating layer may be at least 90 μm to 100 μm.

The device may further comprise a third electrode for use as a reference electrode in the electrochemical cell, wherein the third electrode is coupled to the silicon comprising substrate via a second insulating layer. The second insulating layer does not conduct electricity. It is therefore an electrical insulator. The second insulating layer may conduct heat.

The reference electrode may be referred to by the abbreviation “RE”. The reference electrode may be known as an auxiliary electrode, for example a second auxiliary electrode. The effect of the reference electrode is to improve the function of the electrochemical cell.

The reference electrode may be of any suitable size. The reference electrode may have a longest dimension of about 3.5 mm to about 6mm. The reference electrode may have a longest dimension of about 6mm. The longest dimension may be a length. The reference electrode may have a shortest dimension of about 1 mm to about 2 mm, for example 1.5 mm. The shortest dimension may be a width.

Any suitable material may be used for the reference electrode. The skilled person understands that the reference electrode should be stable during the experiment, not susceptible to corrosion or redox processes in the electrolyte solution or reaction. In a preferred three-electrode electrochemical cell, the reference electrode is isolated from the bulk solution using a glass frit or salt bridge, and the counter electrode is positioned far from the working electrode. However, for reasons of size, cost, and complexity, miniaturized analytical devices may depart from this preferred arrangement. Instead, the third electrode may be a non-isolated “quasi-reference” electrode. The material of the pseudo-reference electrode may be chosen based on the potential window.

The third electrode may comprise silver, gold, platinum or stainless steel. The stainless steel may be wires, sheets or laminated. The third electrode may be a silver electrode. The third electrode may be a Ag/AgCI electrode.

The first insulating layer may correspond to the second insulating layer. For example, they may have the same constituents and/or the same thickness. Alternatively, the first insulating layer may be different to the second insulating layer. For example, they may have different constituents and/or different thicknesses.

In the context of an electrode, “coupled” may refer to a physical, electrical and/or thermal interaction between an electrode and the substrate. Coupled may therefore refer to physically, electrically and/or thermally coupled. Coupling may be direct or indirect. Indirect physical coupling between an electrode and the substrate includes coupling via an intermediate layer. The intermediate layer may be formed on the substrate, formed on the electrode or separately formed. Indirect electrical coupling between an electrode and the substrate includes coupling via an electrically conductive intermediate layer. Indirect thermal coupling between an electrode and the substrate includes coupling via a thermally conductive intermediate layer. Direct coupling between an electrode and the substrate refers to coupling without an intermediate layer. The term “intermediate” is used interchangeably with the term “intervening”. The first insulating layer between the second electrode and the substrate is an example of an intermediate layer. The second insulating layer between the third electrode and the substrate is another example of an intermediate layer.

In the context of the first electrode, coupled includes at least a thermal interaction between the electrode and the substrate. The thermal coupling allows the first electrode to be heated by an electrical current passing through the substrate in use. The first electrode is typically also physically coupled to the substrate. The physical coupling may be direct physical coupling to the substrate. The physical coupling may be direct physical coupling to a porous silicon layer formed on the substrate. The first electrode may also be electrically coupled to the substrate. The electrical coupling may be direct electrical coupling to the substrate. The electrical coupling may be direct electrical coupling to a porous silicon layer formed on the substrate.

In the context of the second and the third electrode, coupling is via the first insulating layer and the second insulating layer, respectively. In the context of the second and/or the third electrode, coupling therefore includes at least an indirect physical interaction with the substrate. Since the insulating layer does not conduct electricity, coupling does not include electrical coupling to the substrate in the context of the second and the third electrode. The second and/or third electrode may be thermally coupled to the substrate via the first insulating layer and the second insulating layer, respectively.

The substrate may comprise one or more porous silicon layers. The one or more porous silicon layers may have around 30 nm to around 60 nm wide pores. The depth of the pores may be around 600 nm.

The first electrode may be coupled to a first surface of the substrate. The first surface of the substrate may be porous silicon. The first surface may be a porous silicon surface. Without being bound by theory, porous silicon may increase the surface area of the substrate available for coupling to the electrode. A porous silicon surface may therefore increase the durability of the device, the ease and/or speed of manufacturing.

The term “Ohmic contact” refers to a non-rectifying electrical junction. The Ohmic contacts may therefore be junctions between two conductors that have a linear current-voltage (I-V) curve as with Ohm's law. Without being bound by theory, low resistance ohmic contacts are used to allow charge to flow easily in both directions between two conductors, without blocking due to rectification or excess power dissipation due to voltage thresholds. The term “ohmic contact” typically refers to an ohmic contact of a metal to a semiconductor. The semiconductor may be silicon. The metal may be any suitable metal. The Ohmic contacts may be metal. Alternatively, the Ohmic contacts may instead be referred to as “metal contacts”. Alternatively, the Ohmic contacts may instead be referred to as “electrical contacts”. In any embodiment described herein the Ohmic contacts may instead be replaced with non-Ohmic contacts. A junction or contact that does not demonstrate a linear I-V curve is called non-ohmic.

In the context of Ohmic contacts, “coupled” refers at least to an electrical interaction. Typically, it also refers to a physical interaction between an Ohmic contact and the substrate. It may also refer to a thermal interaction between an Ohmic contact and the substrate. Coupled may therefore refer to electrically coupled and optionally physically and/or thermally coupled. Coupling may be direct or indirect. Indirect physical coupling between an Ohmic contact and the substrate includes coupling via a suitably electrically conductive intermediate layer. The intermediate layer may be formed on the substrate, formed on the electrode or separately formed. Indirect electrical coupling between an Ohmic contact and the substrate includes coupling via an electrically conductive intermediate layer. Indirect thermal coupling between an Ohmic contact and the substrate includes coupling via a thermally conductive intermediate layer. Direct coupling between an Ohmic contact and the substrate refers to coupling without an intermediate layer. The term “intermediate” is used interchangeably with the term “intervening”.

The Ohmic contacts may be coupled to a second surface of the substrate. The second surface of the substrate may be porous silicon. The second surface may be a porous silicon surface. Without being bound by theory, porous silicon may increase the surface area of the substrate available for coupling to the Ohmic contacts. A porous silicon surface may therefore increase the durability of the device, the ease and/or speed of manufacturing.

The Ohmic contacts may comprise any suitable electrically conductive material. They may be instead be referred to as electrodes or terminals accordingly. The electrically conductive material may be a metal or carbon. For example, the Ohmic contacts may comprise aluminium, copper, lead, tungsten, gold, platinum, iridium, ruthenium, glassy carbon, carbon black, graphite or graphene. The Ohmic contacts may comprise a metal or a metal alloy. In one embodiment, the Ohmic contacts comprise gold. The Ohmic contacts may be gold Ohmic contacts. In a preferred embodiment, the Ohmic contacts comprise aluminium and copper. The Ohmic contacts may be aluminium and copper Ohmic contacts. In a preferred embodiment, the Ohmic contacts comprise aluminium with around 2% to around 4% copper (by weight). The Ohmic contacts may be aluminium with around 2% copper (by weight) Ohmic contacts. The use of aluminium with around 2% copper may prevent electromigration.

A suitable Ohmic contact material may be selected according to the following criteria:

1. low contact resistance to both N+ and P+ regions

2. Ease of formation (deposition, etching)

3. Compatibility with Si processing (cleaning etc.)

4. No diffusion of the contact metal in Si or SiO₂

5. No unwanted reaction with Si or SiO₂ and optionally other materials used in backend technology.

6. No impact on the electrical characteristics of the shallow junction

7. Long term stability.

Aluminium contacts generally fulfil these criteria. The addition of copper in a 2-4% range increases the lifetime and conductivity of the contact.

The Ohmic contacts may correspond to one another. For example, they may have the same constituents and/or the same thickness. Alternatively, the Ohmic contacts may be different to the one another. For example, they may have different constituents and/or different thicknesses.

The Ohmic contacts may correspond to the first electrode. For example, the Ohmic contacts and the first electrode may have the same constituents and/or the same thickness. Alternatively, the Ohmic contacts and the first electrode s may be different to the one another. For example, they may have different constituents and/or different thicknesses.

The Ohmic contacts and/or the first electrode may be around 150 nm thick. They may be described as a 150 nm thick layer. The Ohmic contacts and/or the first electrode may have 40 to 80 nm wide pores. The Ohmic contacts and/or the first electrode may be around 150 nm thick with 40 to 80 nm wide pores. This improves sensitivity due to the high electrochemically active surface area which is two times greater than the geometric area estimated using the Randles—Sevcik equation with experimentally measured gradient values of 0.71±0.08.

The effect of the Ohmic contacts is to enable the substrate to function in use as a Joule heater (also termed a thermal block) and as a thermal sensor (also termed a thermistor).

Joule heating, also known as resistance heating and Ohmic heating, is the process by which the passage of an electric current through a conductor produces heat.

Known devices may perform thermal sensing through pure metals, called resistance temperature detectors (RTD). The device of the present invention uses a thermistor. Thermistors differ from resistance temperature detectors (RTDs) in that the material used in a thermistor is generally a ceramic or polymer, while RTDs use pure metals. The temperature response is also different; RTDs are useful over larger temperature ranges, while thermistors typically achieve a greater precision within a limited temperature range, typically —90° C. to 130° C.

The Ohmic contacts coupled to the silicon comprising substrate and configured to pass a current through the silicon comprising substrate when connected to a power source may be termed Ohmic contacts coupled to the silicon comprising substrate and configured to provide an integrated joule heater and thermal sensor. The Ohmic contacts coupled to the silicon comprising substrate and configured to pass a current through the silicon comprising substrate when connected to a power source may be termed a diode.

The Ohmic contacts and first electrode may be on opposites sides of the substrate. The first and second surfaces of the substrate are typically on opposite sides of the substrate. This allows the electrochemical cell to be physically separated from the Joule heater and thermal sensor functions and enables more convenient interfacing with electrical circuitry relating to each function.

The first surface of the substrate may correspond to the second surface of the substrate. For example, they may have the same constituents and/or the same thickness. Alternatively, the first surface of the substrate may be different to the second surface of the substrate. For example, they may have different constituents and/or different thicknesses. They may each independently be porous silicon.

The first electrode may be positioned between the Ohmic contacts. This may be the case even when the Ohmic contacts and first electrode are on opposites sides of the substrate. “Between” means between with respect to a long axis of the substrate. Without being bound by theory, this positioning may improve the efficiency of heating the first electrode in use.

The distance between the Ohmic contacts may be any suitable distance. The distance between the Ohmic contacts may be up to around 5 mm. The distance between the Ohmic contacts may be a maximum of 5mm. The Ohmic contacts may be up to 5 mm apart. Typically the Ohmic contacts are up to 5 mm apart when the substrate has one or more dimensions of 10 mm. The distance between the Ohmic contacts may be half of one or more dimensions of the substrate.

The first electrode may be in electrical and thermal contact with the substrate.

The first electrode may be electrically insulated from the Ohmic contacts. The substrate may comprise an electrically insulating layer accordingly. The electrically insulating layer may comprise silicon dioxide. The electrically insulating layer may have a thickness from about 100 nm to about 10 μm. The electrically insulating layer may have a thickness of up to about 1 μm. Without being bound by theory, the electrically insulating layer may improve the function of the device by electrically isolating the electrochemical cell from the Joule heater and thermal sensor functions.

This may in turn reduce interference in use.

The device may further comprise a chamber for retaining the solution, wherein the first electrode is positioned within the chamber. The second and/or third electrode may also be positioned within the chamber. The Ohmic contacts may be positioned outside the chamber. This allows the electrochemical cell to be physically separated from the Joule heater and thermal sensor functions and enables more convenient interfacing with electrical circuitry relating to each function.

The chamber is in thermal contact with the substrate. This enables the solution in the chamber to be heated when a current is passed through the substrate via the Ohmic contacts in use.

The chamber may be any suitable container. The chamber may have any suitable volume. The chamber volume and shape may be adapted to ensure the electrochemical cell is covered in the solution in use. The chamber may have a volume ranging from about 20 μl to about 40 μl.

The chamber may also be termed a reaction chamber. The chamber may retain a solution while it undergoes a reaction, in use. For example, where the substance is a nucleic acid, the solution may undergo nucleic acid amplification in use. The thermal contact between the substrate and the reaction chamber may allow control of the temperature of the solution in the reaction chamber. For example, the thermal contact may allow the temperature of the solution to be held at an appropriate temperature to perform a step of the polymerase chain reaction (PCR). The chamber may prevent the evaporation of the solution under DNA amplification conditions.

The solution may be a reaction mixture. The reaction mixture may be any suitable medium in which a desired chemical reaction may take place. For example, the reaction mixture may be any suitable medium in which amplification of a nucleic acid may take place. The reaction mixture may be a liquid medium. The reaction mixture may comprise deionised water. The reaction medium may comprise a PCR buffer, an isothermal buffer, MgSO₄, BSA, Betaine, DMSO, DTT, Tween, PEG and/or Syto9. The reaction mixture may be of any suitable volume. The optimal volume of reaction mixture may be determined by the skilled person, accounting for example for the size of the chamber.

Since the Ohmic contacts are configured to pass a current through the substrate when connected to a power source they may be described as being configured to heat the substrate when connected to a power source.

The power source may be a variable power source. The power source may be a battery or mains power.

Non-limiting examples of electrode combinations which may be used include:
An indium tin oxide working electrode, a platinum reference electrode and a platinum counter electrode.
A platinum working electrode, a gold reference electrode and a gold counter electrode.
A gold working electrode, a Ag/AgCI reference electrode and a gold counter electrode.
A platinum working electrode, a platinum reference electrode and a platinum counter electrode.
A platinum working electrode, a platinum reference electrode and a platinum counter electrode.
A porous silicon comprising an immobilised enzyme working electrode, a Ag/AgCI reference electrode and a platinum counter electrode.
A gold nanostructured porous silicon working electrode, a silver reference electrode and a platinum counter electrode.
A Polypyrrole (PPy) film working electrode, a Ag/AgCI reference electrode and a platinum counter electrode.

According to a second aspect, the invention provides a system for detecting a substance in a solution, the system comprising:
a device according to the first aspect of the invention; and one or more of:
   a potentiostat configured to control an electrochemical potential of the first electrode;
   a power source configured to pass a current through the substrate via the Ohmic contacts; and
   a control unit connected to the Ohmic contacts and configured to calculate a resistance of the substrate.

The potentiostat is an electrochemical reader which may be connected to the first electrode and optionally to the second and/or third electrodes. The connection may be made by any suitable means, for example by wires and crocodile clamps. Suitable potentiostats are known in the art. One suitable potentiostat is the “PalmSens3” model from Palmsens, UK. The potentiostat is connected to a computer, so parameters such as potential (applied between the WE and the RE), current, potential step, data acquisition, pulse width, pulse amplitude and/or chronotechnique duration, are controlled and recorded through a software associated to the potentiostat.

A potentiostat is an electronic instrument that controls the voltage difference between a Working Electrode and a Reference Electrode. Both electrodes may be contained in an electrochemical cell. The potentiostat implements this control by injecting current into the cell through an Auxiliary, or Counter, electrode.

Typically, the potentiostat measures the current flow between the Working and Counter electrodes.

The potentiostat may be arranged as a component of a potentiostat circuit. The potentiostat circuit may comprise an electrometer. The electrometer in the potentiostat circuit measures the voltage difference between the reference and working electrodes. Its output has two major functions: it is the feedback signal in the potentiostat circuit, and it is the signal that is measured whenever the cell voltage is needed.

An ideal electrometer has zero input current and an infinite input impedance. Current flow through the reference electrode can change its potential. In practice, all modern electrometers have input currents close enough to zero that this effect can usually be ignored. Two important electrometer characteristics are its bandwidth and its input capacitance. The electrometer bandwidth characterizes the AC frequencies the electrometer can measure when it is driven from a low-impedance source. The electrometer bandwidth may be higher than the bandwidth of the other electronic components in the potentiostat.

The electrometer input capacitance and the reference electrode resistance form an RC-filter. If this filter's time constant is too large, it can limit the effective bandwidth of the electrometer and cause system instabilities. Smaller input capacitance may provide a more stable operation and greater tolerance for high impedance reference electrodes.

The system may further comprise a current-to-voltage (WE) converter. The current-to-voltage (I/E) converter measures the cell current. The system may further comprise a current-measurement resistor, Rm. The current-to-voltage (I/E) converter may force the cell current to flow through the current-measurement resistor. The voltage drop across Rm is a measure of the cell current

The system may comprise a plurality of current-measurement resistors. A plurality of Rm may be advantages in applications where the current is variable. For example, in a corrosion experiment, the current can often vary by as much as seven orders of magnitude. In such cases, more than a single resistor is needed to measure current. A number of different Rm resistors can be automatically switched into the I/E circuit. This allows measurement of widely varying currents, with each current measured using an appropriate resistor. An “I/E autoranging” algorithm may be used to select the appropriate resistor values. The I/E converter's bandwidth depends on its sensitivity. Measurement of small currents requires large Rm values. Stray (unwanted) capacitance in the I/E converter forms an RC-filter with Rm, limiting the I/E bandwidth. No potentiostat can accurately measure 10 nA at 100 kHz because the bandwidth on this current range is too low to measure a frequency of 100 kHz.

This effect may be especially important in electrochemical impedance spectroscopy (EIS) measurements This is because in EIS the frequency is typically swept from 0.1 Hz to high frequencies (usually 100 kHz) while in other electrochemical techniques, like square-wave voltammetry, the maximum frequency can be 100 Hz, so this effect is not expected to be relevant to other techniques.

The system may comprise a control amplifier. The control amplifier may be a servo amplifier. It compares the measured cell voltage with the desired voltage and drives current into the cell to force the voltages to be the same. The measured voltage is input into the negative input of the control amplifier. A positive perturbation in the measured voltage creates a negative control amplifier output. This negative output counteracts the initial perturbation. This control scheme is known as negative feedback. Under normal conditions, the cell voltage is controlled to be identical to the signal source voltage. The control amplifier may have a limited output capability. For example, in the case of the Gamry Instruments' Reference 3000, the control amplifier cannot output more than 32 V or more than 3 A.

The system may comprise a signal circuit. The signal circuit may be a computer-controlled voltage source. It is typically the output of a Digital-to-Analog (D/A) converter that converts computer-generated numbers into voltages. Proper choice of number sequences allows the computer to generate constant voltages, voltage ramps and sine waves at the signal circuit's output. When a D/A converter is used to generate a waveform such as a sine wave or a ramp, the waveform may be a digital approximation of the equivalent analog waveform, with small voltage steps. The size of these steps is controlled by the resolution of the D/A converter and the rate it at which it is being updated with new numbers.

The control unit may be any suitable unit capable of calculating the resistance of the substrate through connection to the Ohmic contacts. The connection may be made by any suitable means, for example by wires and crocodile clamps. Resistance is typically calculated using Ohm's law based on measuring the voltage passed through the Ohmic contacts and the substrate when a constant current is applied. The skilled person can select a constant current to be applied for thermal sensing that is small enough to no produce a significant change of temperature in the device. The constant current may be around 5 mA to around 40 mA. The constant current may be around 10 mA.

The electrochemical cell may be connected to an electrochemical reader outside the reaction chamber.

According to a third aspect, the invention provides a method of fabricating a device according to the first aspect of the invention comprising
electroplating the first electrode to the substrate, thereby coupling the first electrode to the substrate, and
electroplating the Ohmic contacts to the substrate thereby coupling the Ohmic contacts to the substrate.

Fabricating means making, assembling or manufacturing.

Electroplating may be in an electroplating solution. The electroplating solution may comprise KAu(CN)₂, KAuCl₄, KAuCl₃, KAuCl₂, Na_{3[}Au(S₂O₃)_{2]}, K₂HPO₄, KH₂PO₄, K₂CO₃ and/or KCN. The electroporating solution may comprise KAu(CN)₂ or KCN. Any suitable concentration may be used. By way of example, the concentration of the KAu(CN)₂ or KCN may be from around 1 mM to around 100 mM, such as around 10mM. The KAuCl₄, KAuCl₃, KAuCl₂ may be in diluted HCl (e.g. 0.1 M). The skilled person knows to increase the plating time for lower concentrations. Likewise, the skilled person knows to decrease the plating time for higher currents. The current may be from around 10 mA to around 30 mA. Electroplating the first electrode and/or electroporating the Ohmic contacts may for example be in a 10 mM KAu(CN)₂/KCN aqueous bath applying 10 mA versus Pt wire.

Electroplating may comprise pre-electroplating cleaning. The pre-electroplating cleaning may be with an HF solution. The HF solution may be a 5% HF solution. The cleaning with HF is not essential but may improve the metal electroplating. Without being bound by theory, the cleaning may remove native oxide from the surface of the silicon substrate and produce an electrically conductive surface.

In an alternative embodiment, the first electrode, second electrode and/or third electrode may be screen printed to the substrate. The Ohmic contacts may be screen printed to the substrate. The Ohmic contacts, the first electrode, second electrode and/or third electrode may be screen printed to the substrate.

The first electrode and the Ohmic contacts may be simultaneously coupled to the substrate by electroplating. In this embodiment, the Ohmic contacts and the first electrode may have the same constituents and/or the same thickness.

The method may further comprise forming one or more porous silicon layers on the substrate, prior to the electroplating. The porous silicon layer may be formed by Metal-assisted chemical etching (MACE), anodization, galvanization, photoetching, HNO₃/HF vapor etching, by mechanical means or by stain-etching. Typically, the porous silicon layer may be formed by Metal-assisted chemical etching (MACE). The advantages of using metal-assisted etching to obtain PSi are numerous. The method is easy to handle, suitable for batch fabrication of porous Si devices and PSi layers can be formed on highly resistive Si. Compared to stain-etched layers, those obtained by MACE have better uniformity and much higher thickness.

The method may therefore be a method of fabricating a device according to the first aspect of the invention comprising
forming a porous silicon layer or layers on the substrate, optionally by Metal-assisted chemical etching (MACE),
electroplating the first electrode to the substrate, thereby coupling the first electrode to the substrate, and
electroplating the Ohmic contacts to the substrate thereby coupling the Ohmic contacts to the substrate.

The substrate may be formed from a silicon wafer. Any type of silicon wafer may be used. The silicon wafer may be a p-type silicon wafer. Typically, the silicon wafer is a pristine p-type silicon wafer.

The method may further comprise an initial step of cleaning the silicon wafer. The silicon wafer may be cleaned with a solvent such as acetone. After cleaning with a solvent the silicon wafer may be rinsed with distilled water.

The silicon wafer may be cleaned using a piranha surface treatment. This may be an alternative or additional to cleaning with a solvent. The piranha surface treatment may comprise immersing the silicon wafer in a piranha solution. The piranha solution may comprise H₂SO₄ and/or H₂O₂. The piranha solution may be 95% H₂SO_{4/}30% H₂O₂ (v/ v).

The MACE may use a catalyst selected from the group consisting of Gold, Platinum, Silver, Nickel, Manganese, Cobalt, Copper, Chromium, Magnesium and Iron. The catalyst may be gold, silver or platinum. The catalyst is typically gold.

The MACE may be two-step MACE. The first step of two-step MACE is electroless plating. The second step of two-step MACE is the production of silicon nanowires. The second step results in the production of silicon nanowires and may alternatively be termed “etching”.

The method may therefore be a method of fabricating a device according to the first aspect of the invention comprising
optionally cleaning a silicon wafer to form the substrate;
forming a porous silicon layer or layers on the substrate by two-step Metal-assisted chemical etching (MACE), optionally using gold as a catalyst, the two-step MACE comprising:
   a) electroless plating, and
   b) producing silicon nanowires;
electroplating the first electrode to the substrate, thereby coupling the first electrode to the substrate, and
electroplating the Ohmic contacts to the substrate thereby coupling the Ohmic contacts to the substrate.

The electroless plating may comprise metal sputtering or immersing the substrate an electroless plating solution. The electroless plating may be with gold particles. The electroless plating may be in a solution comprising KAuCl₄ and/or hydrogen fluoride (HF). The solution may be an aqueous solution. The KAuCl₄ may be at a concentration of around 10 μM to around 100 μM, for example around 80 μM KAuCl₄. The HF may be at around 0.5% v/v. The electroless plating solution may be an 80 μM KAuCl₄ and 0.5% HF aqueous solution. The electroless plating may be for from around 10 seconds to around 30 seconds, for example around 20 seconds.

The substrate may be rinsed and dried before etching. Rinsing may be in distilled water.

The etching may comprise immersing the substrate an etching solution. The etching may be in a solution comprising hydrogen peroxide (H₂O₂) and/or hydrogen fluoride (HF). The ratio of H₂O₂ to HF may be 3:1. The etching solution may be diluted in distilled water. The dilution may be 1:20. The etching solution may be 1:20 (30% H₂O₂: 10% HF v/v). The etching may be for around 7 to 10 minutes. Typically, the etching is for around 10 minutes.

After forming a porous silicon layer or layers, the substrate may be cut to the desired size of the device. Cutting may alternatively occur at any other suitable point in the method, for example prior to forming porous silicon layers. However, cutting after forming porous silicon layers typically is most efficient. The substrate may be cut into squares. The squares may for example be around 1 cm×1 cm.

The first electrode and the Ohmic contacts typically do not cover the entire surface of the substrate. The method may comprise transferring a pattern onto the substrate prior to electroplating the first electrode and/or the Ohmic contacts. The pattern may define one or more shielded surfaces on the substrate. A shielded surface may be an area where electroplating onto the substrate does not occur. Accordingly, the shielded surface(s) may define the location of one or more other components to be coupled to the substrate by subsequent method steps. For example, the shielded surface(s) may define the location at which the second and/or third electrode are coupled to the substrate.

The method may therefore be a method of fabricating a device according to the first aspect of the invention comprising
optionally cleaning a silicon wafer to form the substrate;
forming a porous silicon layer or layers on the substrate by two-step Metal-assisted chemical etching (MACE), optionally using gold as a catalyst, the two-step MACE comprising:
   a) electroless plating, and
   b) producing silicon nanowires;
transferring a pattern onto the substrate to define a shielded area;
electroplating the first electrode to the substrate, thereby coupling the first electrode to the substrate, and
electroplating the Ohmic contacts to the substrate thereby coupling the Ohmic contacts to the substrate.

The pattern may be transferred onto the substrate by affixing a non-conductor to the surface. The pattern may therefore alternatively be termed a “non-conductor” or a “shield”. The pattern may comprise a thermoplastic material. The thermoplastic material may comprise polyethylene terephthalate (PET)-Polyethylene (PE).

The pattern may be transferred to the surface by heat pressing. The heat pressing may be at from around 120° C. to around 200° C. For example, the heat pressing may be at around 180° C. The skilled person will understand the higher the temperature, the shorter the heating time and can adjust either parameter accordingly. The heat pressing may be for around 5 minutes. The heat pressing may be for around 2 minutes. The present inventors have found the best resolution is obtained at 180° C. for 5 min for PET-PE coupling and at 180° C. for 2 min for RE/CE-PET coupling.

The pattern may be transferred to multiple surfaces of the substrate simultaneously. The pattern may be transferred to a first and second surface of the substrate simultaneously. The pattern may define a shielded area to which the second and/or third electrode will be coupled.

This thermoplastic material fills determinate PSi regions, defining the geometry of the subsequent gold-plated electrodes and preventing any further inhibition effect of non-passivated PSi during the nucleic acid amplification by PCR.

Electroplating may therefore couple the first electrode and/or the Ohmic contacts to one or more non-shielded areas of the substrate.

The pattern may be transferred onto the substrate by affixing a non-conductor bonded to a conductor to the surface, wherein the non-conductor contacts the substrate. The non-conductor may be a thermoplastic material. The thermoplastic material may comprise mPPE (modified polyphenylene ether), polyethylene terephthalate (PET), thermoplastic elastomer (TPE) or polyethylene (PE). The thermoplastic material may comprise polyethylene terephthalate (PET)-Polyethylene (PE). The conductor may be a metal, such as platinum, copper, or stainless steel. The non-conductor bonded to a conductor may be termed a conductor laminated non-conductor. For example, the non-conductor bonded to a conductor may be a copper-laminated PET-PE (Cu-PET-PE). The non-conductor bonded to a conductor may be a silver-plated copper-laminated PET-PE (AG@Cu-PET-PE). In this case, the PET-PE substrate facilitates the physical bonding of the metal layer with the former PET-PE layer on PSi and acts as electrical insulator between the RE/CE and WE electrodes.

A chamber may be affixed to the surface coupled to the first electrode. The chamber may comprise glass and/or any polymer stable at 100° C. and above. The chamber may comprise polyethylene. The chamber may comprise two holes. The chamber may define the analytical zone (around 5-mm diameter) and volume (around 20-40 μl) but, more importantly, prevents the evaporation of the solution under DNA amplification conditions.

The chamber may be affixed by partially melting the base of the chamber. For example, in the case of a polyethylene chamber, the base of the chamber may be partially melted by applying a current equivalent to 110±10° C. between the Ohmic contacts.

The device of the invention may be manufactured through wet chemistry and optionally thermal bonding, consequently negating the need of a cleanroom or time-consuming expensive equipment such as plasma deposition tools. The method of fabrication therefore has the advantage of using less time and being less costly than known methods. Since less specialised equipment and expertise is needed, the device may therefore be fabricated closer to the point of need. This may be critical during disease outbreaks when usual routes of transportation and supply may be impaired.

The device may be fabricated at wafer-scale in a standard laboratory (no cleanroom processing required). Therefore, the device may be low-cost; A 4-inch Si wafer containing 37 microsensors of 10×10×0.6 mm in size and can be produced in 7 hours, costing ˜0.33 GBP per microsensor.

According to a fourth aspect, the invention provides a method of detecting a substance in a solution using a device according to the first aspect of the invention, the method comprising
placing the solution in contact with the first electrode,
passing a current through the substrate, and
detecting a substance in the solution.

The method may further comprise maintaining a temperature to allow one or more steps of the polymerase chain reaction (PCR) to occur. The method may further comprise maintaining a temperature to allow isothermal nucleic acid amplification to occur.

Passing a current through the substrate may have the effect of holding the solution at a temperature. The temperature may be a temperature required for a biochemical reaction. The biological reaction may be PCR or isothermal nucleic acid amplification. The current and/or the temperature may be controlled by reading the changes in the resistance of the substrate using a control unit. The changes in the resistance of the substrate may be measured via the Ohmic contacts.

The method may further comprise applying a current to the first electrode. The method may further comprise operating the electrochemical cell.

In use, the device and/or the solution may be heated. Different currents may be applied to the Ohmic contacts corresponding to the temperature required. The current may be a constant current. The current may be applied around every 0.25 seconds to around every 1 second. The current may be applied around every 0.5 seconds. The current may be from around 10 mA to around 50 mA. The current may be around 10 mA. For example, every 0.5 seconds a constant current of about 10 mA may be applied. Without being bound by theory, the application of a constant current for a relatively short period, such as around 1 second or less, improves the accuracy of heating to a desired temperature. The voltage passed through the Ohmic contacts may be measured by the controller. The resistance may be calculated using Ohm's law. The calculation may be performed by software. The calculation may therefore be a computer implemented calculation.

The method may comprise cycling through the three steps of PCR. Accordingly the method may comprise:
An optional initiation step. The initiation step is used with DNA polymerases that require heat activation by hot-start PCR. The initiation step may comprise heating the solution to around 94 to around 98 degrees Celsius for around 1 minute to around 10 minutes.
A denaturation step. The denaturation step may comprise heating the solution to around 94 to around 98 degrees Celsius for around 20 seconds to around 30 seconds.
An annealing step. The annealing step may comprise cooling the solution to around 50 to around 68 degrees Celsius for around 20 to around 40 seconds, such as for 30 seconds.
An extension step. The extension step may comprise heating the solution to around 75 to around 80 degrees Celsius, such as around 72 degrees Celsius for around 60 seconds.
An optional final elongation step. The final elongation step may comprise heating the solution to around 70 to around 74 degrees Celsius for around 5 to around 15 minutes.
An optional final hold step. The final hold step may comprise allowing the solution to cool to around 4 to around 15 degrees Celsius for an indefinite time. The final hold step may allow short-term storage of PCR products.

The denaturation, annealing and extension steps may be repeated around 20 to around 40 times, for example around 35 times. Each repeat of the denaturation, annealing and extension steps may together be termed a cycle.

The method may further comprise an electroanalysis step. The electroanalysis step may comprise heating the solution to around 30 to 50 degrees Celsius, such as around 40 degrees Celsius, for around 30 seconds to around 2 minutes. The electroanalysis step may be performed at room temperature. The electroanalysis step may be performed without applying any current.

Isothermal amplification and PCR amplification temperatures (40-95° C.) may be reached by applying from around 150 mA to around 450 mA electrical currents to the Ohmic contacts. The method may therefore comprise passing a current of around 150 mA to around 450 mA through the substrate. The method may comprise passing a current of around 150 mA to around 350 mA through the substrate. Temperatures higher than 120° C. (corresponding to the application of more than 450 mA) may degrade the DNA and/or the electrochemical cell, for example due to the melting of the PET-PE layers. This linear temperature-current response suggests that the Ohmic contacts, such as a Au—Si—Au diode, behaves as ohmic resistor under the current and voltage conditions required for 25-100° C. Joule heating.

The passing a current through the substrate may be passing a sequence of currents through the substrate. The sequence of currents may be termed a succession of currents or a program of currents. A sequence of currents suitable for performing a PCR cycle is:
i) denaturation (94° C.): 410 mA for 23 s and 400 mA for 30 s;
ii) primer annealing (63° C.): 0 mA for 10 s and 270 mA for 30 s;
iii) extension (72° C.): 315 mA for 3 s and 310 mA for 30 s;
iv) electroanalysis (40° C.): 0 mA for 12 s and 170 mA for 30 s.

Without being bound by theory, the first current and time values of each phase brings the solution to the temperature required for the PCR cycle. The second current and time values of each phase are related to the PCR cycle. The second current value for each phase may vary by around ±20 mA. The second time value may be from around 15 seconds to around 60 seconds.

The sequence of currents may therefore comprise:
(i) a first current of around 410 mA for around 23 seconds and a second current of around 380 mA to around 420 mA for around 15 to around 60 seconds;
(ii) an optional third current of around 0 mA for around 10 seconds and a fourth current of around 250 mA to around 290 mA for around 15 to around 60 seconds;
(iii) a fifth current of around 315 mA for around 3 seconds and a sixth current of around 290 to around 330 mA for around 15 to around 60 seconds;
(iv) a optional seventh current of around 0 mA for around 10 to 15 seconds and an optional eighth current of less than or equal to around 190 mA for around 30 seconds.

Since the third current is of around 0 mA, the third current is an optional third current. The third current may be a phase where no current is applied.

Since the seventh current is of around 0 mA, the seventh current is an optional seventh current. The seventh current may be a phase where no current is applied.

The sequence of currents may comprise:
(i) a first current of around 410 mA for around 23 seconds and a second current of around 400 mA for around 30 seconds;
(ii) a third current of around 0 mA for around 10 seconds and a fourth current of around 270 mA for around 30 seconds;
(iii) a fifth current of around 315 mA for around 3 seconds and a sixth current of around 310 mA for around 30 seconds;
(iv) a seventh current of around 0 mA for around 12 seconds and an eighth current of around 170 mA for around 30 seconds.

The isothermal nucleic acid amplification may be selected from the group consisting of loop-mediated isothermal amplification (LAMP), strand displacement amplification (SDA), helicase-dependent amplification (HDA) and nicking enzyme amplification reaction (NEAR). Isothermal nucleic acid amplification typically comprises a single amplification step. The amplification step may be at around 60 to around 65° C. Isothermal amplification may be followed by an electroanalysis step.

A sequence of currents suitable for performing LAMP is:
i) amplification at around 60 to around 65° C.: a first current of around 270 mA for around 10 seconds and a second current of around 260±20 mA for around 30 seconds to around 5 minutes
ii) electroanalysis at around 40° C.: a third current of around 0 mA for around 10 to 15 seconds and an optional fourth current of less than or equal to around 190 mA for around 30 seconds. Since the third current is of around 0 mA, the seventh current is an optional third current. The third current may be a phase where no current is applied.

The sequence of currents may therefore comprise:
(i) a first current of around 270 mA for around 10 seconds and a second current of around 240 mA to around 280mA for around 30 seconds to around 5 minutes
(ii) a third current of around 0 mA for around 10 to 15 seconds and an optional fourth current of less than or equal to around 190 mA for around 30 seconds.

The solution may further comprise any suitable primers able to anneal to a target nucleic acid under stringent conditions. The solution may therefore comprise any suitable primers needed for amplification to take place. The solution may further comprise a DNA polymerase, such as a Taq polymerase. The solution may further comprise a buffer solution. The solution may further comprise bivalent cations, such as Mg²⁺ or Mn²⁺. The solution may further comprise monovalent cations such as K⁺ ions.

An electrochemical approach for the detection of DNA may the use of methylene blue (MB). The solution may further comprise an electrochemical reporter, such as MB. MB is a redox-active reporter that is intercalated between guanine-cytosine base pairs of the double-strand DNA (ds-DNA). Intercalation of molecules MB into the ds-DNA reduce the concentration of free MB in solution, leading to a decreased redox signal during electroanalysis. The method may therefore further comprise detecting a decreased signal, such as a redox signal, during electroanalysis. The method may further comprise detecting a substance in the solution when a decreased signal is detected during electrolysis.

As used herein, “stringent conditions” are known to those skilled in the art and can be found in Current Protocols in Molecular Biology, John Wiley & Sons, N.Y., 6.3.1-6.3.6, 1991. The stringent conditions may be stringent conditions for hybridisation. The stringent conditions may be stringent conditions for annealing. Stringent conditions may be defined as equivalent to annealing in isothermal buffer. Isothermal buffer (1×) may comprise 20 mM Tris-HCl; 10 mM (NH₄)₂SO₄; 50 mM KCl; 8 mM MgSO₄; Betaine 0.8M; 0.1% Tween® 20; and may have pH 8.8 at 25° C.

As used herein, “nucleic acid sequence” may refer to either a double stranded or to a single stranded nucleic acid molecule. The nucleic acid sequence may therefore alternatively be defined as a nucleic acid molecule. The nucleic acid molecule comprises two or more nucleotides. The nucleic acid sequence may be synthetic. The nucleic acid sequence may refer to a nucleic acid sequence that was present in the sample on collection. Alternatively, the nucleic acid sequence may be an amplified nucleic acid sequence or an intermediate in the amplification of a nucleic acid sequence, such as a dumbbell shaped intermediate or a stem-loop intermediate, which are described further below.

As used herein, “anneal”, “annealing”, “hybridise” and “hybridising” refer to complementary sequences of single-stranded regions of a nucleic acid pairing via hydrogen bonds to form a double-stranded polynucleotide. As used herein, “anneal”, “anneals”, “hybridise” and “hybridises” may refer to an active step. Alternatively, as used herein, “anneal”, “anneals”, “hybridise” and “hybridises” may refer to a capacity to anneal or hybridise; for example, that a primer is configured to anneal or hybridise and/or that the primer is complementary to a target. Accordingly, for example, a reference to a primer or a region of a primer which anneals to a nucleic acid sequence or a region of a nucleic acid sequence may in a method of the invention mean either that the annealing is a required step of the method; that the primer or region of the primer is complementary to the nucleic acid sequence or region of the nucleic acid sequence; or that the primer or region of the primer is configured to anneal to the nucleic acid sequence or region of the nucleic acid sequence.

The term “primer” as used herein refers to a nucleic acid, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e. in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the nucleic acid primer typically contains 15 to 25 or more nucleotides, although it may contain fewer or more nucleotides. According to the present invention a nucleic acid primer typically contains 13 to 30 or more nucleotides.

The device of the invention can be more easily used by untrained personnel than those utilising fluorescence-based detection because electrochemical reactions are less susceptible to contamination than fluorescence-based detection.

The method of the invention may be performed in a number of repeats. For example, the method may be performed in duplicate or triplicate.

According to a fifth aspect, the invention provides a method of diagnosis using a device according to the first aspect of the invention, the method comprising
placing a sample in contact with the first electrode,
passing a current through the substrate, and
detecting a substance in the sample.

The method may be an in vitro or an ex vivo method. The method of diagnosis may comprise any of the steps disclosed in connection with the fourth aspect of the invention, mutatis mutandis.

The sample may be a biological sample or a chemical sample.

The chemical sample may be any suitable sample comprising a chemical molecule.

The sample may be a sample from a subject.

As used herein, the terms “subject” and “patient” are used interchangeably to refer to a human or a non-human mammal. The subject may be a companion non-human mammal (i.e. a pet, such as a dog, a cat, a guinea pig, or a non-human primate, such as a monkey or a chimpanzee), an agricultural farm animal mammal, e.g. an ungulate mammal (such as a horse, a cow, a pig, or a goat) or a laboratory non-human mammal (e.g., a mouse and a rat). The invention may find greatest application in connection with the human subjects. In any of the embodiments herein, the subject may be a human.

The sample may be any suitable sample comprising a biological molecule. For example, the sample may comprise a nucleic acid. The sample may be an environmental sample or a clinical sample. The sample may also be a sample of synthetic DNA (such as gBlocks) or a sample of a plasmid. The plasmid may include a gene or gene fragment of interest.

The environmental sample may be a sample from air, water, animal matter, plant matter or a surface. An environmental sample from water may be salt water, brackish water or fresh water. For example, an environmental sample from salt water may be from an ocean, sea or salt marsh. An environmental sample from brackish water may be from an estuary. An environmental sample from fresh water may be from a natural source such as a puddle, pond, stream, river, lake. An environmental sample from fresh water may also be from a man-made source such as a water supply system, a storage tank, a canal or a reservoir. An environmental sample from animal matter may, for example, be from a dead animal or a biopsy of a live animal. An environmental sample from plant matter may, for example, be from a foodstock, a plant bulb or a plant seed. An environmental sample from a surface may be from an indoor or an outdoor surface. For example, the outdoor surface be soil or compost. The indoor surface may, for example, be from a hospital, such as an operating theatre or surgical equipment, or from a dwelling, such as a food preparation area, food preparation equipment or utensils. The environmental sample may contain or be suspected of containing a pathogen. Accordingly, the nucleic acid may be a nucleic acid from the pathogen.

The clinical sample may be a sample from a patient. The nucleic acid may be a nucleic acid from the patient. The clinical sample may be a sample from a bodily fluid. The clinical sample may be from blood, serum, lymph, urine, faeces, semen, sweat, tears, amniotic fluid, wound exudate or any other bodily fluid or secretion in a state of heath or disease. The clinical sample may be a sample of cells or a cellular sample. The clinical sample may comprise cells. The clinical sample may be a tissue sample. The clinical sample may be a biopsy.

The clinical sample may be from a tumour. The clinical sample may comprise cancer cells. Accordingly, the nucleic acid may be a nucleic acid from a cancer cell.

The sample may be obtained by any suitable method. Accordingly, the method of the invention may comprise a step of obtaining the sample. For example, the environmental air sample may be obtained by impingement in liquids, impaction on solid surfaces, sedimentation, filtration, centrifugation, electrostatic precipitation, or thermal precipitation. The water sample may be obtained by containment, by using pour plates, spread plates or membrane filtration. The surface sample may be obtained by a sample/rinse method, by direct immersion, by containment, or by replicate organism direct agar contact (RODAC).

The sample from a patient may contain or be suspected of containing a pathogen. Accordingly, the nucleic acid may be a nucleic acid from the pathogen. Alternatively, the nucleic acid may be a nucleic acid from the host.

The pathogen may be any entity comprising a nucleic acid. The pathogen may be a eukaryote, a prokaryote or a virus. The pathogen may be an animal, a plant, a fungus, a protozoan, a chromist, a bacterium or an archaeum.

The pathogen may be from the pylum Ascomycota. The pathogen may be from the class Eurotiomycetes. The pathogen may be from the order Eurotiales. The pathogen may be from the family Trichocomaceae. The pathogen may be from the genus Aspergillus. The pathogen may be Aspergillus fumigatus.

The pathogen may be from the pylum Actinobacteria. The pathogen may be from the order Actinomycetales. The pathogen may be from the suborder Corynebacterineae. The pathogen may be from the family Mycobacteriaceae. The pathogen may be from the genus Mycobacterium.

In one embodiment, the pathogen may be a Coronavirus (CoV). The coronavirus may be SARS-CoV-2. The invention may therefore provide a method for diagnosis of a coronavirus infection and/or disease, such as COVID-19.

In one embodiment, the pathogen may be Tuberculosis. The invention may therefore provide a method for diagnosis of tuberculosis. The tuberculosis may be mycobacterium avium paratuberculosis (MAP). MAP is associated with Johne's disease in livestock. The invention may therefore provide a method for diagnosis of tuberculosis, such as Johne's disease in livestock. The invention may provide a method for diagnosis of paratuberculosis (ParaTB).

The pathogen may be a drug resistant bacteria. The invention may therefore provide a method for diagnosis of a drug resistant bacteria infection.

The nucleic acid may be isolated, extracted and/or purified from the sample prior to use in the method of the invention. The isolation, extraction and/or purification may be performed by any suitable technique. For example, the nucleic acid isolation, extraction and/or purification may be performed using a nucleic acid isolation kit, a nucleic acid extraction kit or a nucleic acid purification kit, respectively.

The method of the invention may further comprise an initial step of isolating, extracting and/or purifying the nucleic acid from the sample. The method may therefore further comprise isolating the nucleic acid from the sample. The method may further comprise extracting the nucleic acid from the sample. The method may further comprise purifying the nucleic acid from the sample. Alternatively, the method may comprise direct amplification from the sample without an initial step of isolating, extracting and/or purifying the nucleic acid from the sample. Accordingly, the method may comprise lysing cells in the sample or amplifying free circulating DNA.

Following isolation, extraction and/or purification, the nucleic acid may be used immediately or may be stored under suitable conditions prior to use. Accordingly, the method of the invention may further comprise a step of storing the nucleic acid after the extracting step and before the amplifying step.

The step of obtaining the sample and/or the step of isolating, extracting and/or purifying the nucleic acid from the sample may occur in a different location to the subsequent steps of the method. Accordingly, the method may further comprise a step of transporting the sample and/or transporting the nucleic acid.

It is possible for the method to be performed at the point-of-care. As used herein “at the point-of-care” means in the same or a nearby location to the place where the sample originates. In other words, it may not be necessary to transport the sample, or the nucleic acid, and/or to perform any of the method steps in a location remote from the location at which the sample was obtained. When the sample is a clinical sample, the point-of-care may be the location of the patient from whom the clinical sample was obtained, or a location nearby. When the sample is an environmental sample, the point-of-care may be the location of the air, water, animal matter, plant matter or a surface from whom the environmental sample was obtained, or a location nearby. The method of the invention may be suitable for use at the point-of-care. Accordingly, the method may be described as a method for detecting a tandem repeat in a nucleic acid sequence at the point-of-care. One, more or all of the method steps may be described as being performed at the point-of-care. The amplifying step may be performed at the point-of-care. The detecting step may be performed at the point-of-care. The step of obtaining the sample and/or the step of isolating, extracting and/or purifying the nucleic acid from the sample may be performed at the point-of-care.

Accordingly, the method may be performed extemporaneously. As used herein “extemporaneously” means as soon as the sample is obtained, without delay after the sample is obtained, without transporting the sample, in the same or nearby location to the place where the sample originates and/or at the point-of-care. The method may be performed extemporaneously on the sample. The method may be performed extemporaneously by the same individual who took the sample. For example, the method may be performed extemporaneously by the medical practitioner who took the sample.

The substance, such as the nucleic acid, may be a biomarker for a disease or an infection. The method may therefore be defined as a method for diagnosing a disease or an infection comprising detecting a substance in a solution according to the fourth aspect of the invention.

A “biomarker” is a naturally occurring molecule, gene, or characteristic by which a particular pathological or physiological process, disease, etc. can be identified. A biomarker may be a measurable indicator by which a particular pathological or physiological process, disease, etc. can be identified. Accordingly, a biomarker may be a measurable indicator of the presence of an infectious disease or drug resistant infection. The biomarker may increase or decrease in concentration in a sample when the infectious disease or drug resistant infection is present. For example, a tandem repeat may be present at a higher concentration when the sample is from a subject with an infectious disease or drug resistant infection than in a control sample. The relevant control sample may be from a different subject. Alternatively, the control sample may a different sample from the same subject, such as a sample from another location or time point. The other location may be, for example, a non-infected region or a different infected region of the same subject. The other time point may for example be an earlier or a later time point when the infectious disease or drug resistant infection was not present and/or not symptomatic.

The method may further comprise diagnosing a disease or an infection if the substance is present. The method may further comprise diagnosing a disease or an infection if the substance is detected.

The infectious disease may be caused by one or more pathogenic microorganisms, such as bacteria, viruses, parasites or fungi. Infectious diseases can be spread, directly or indirectly, from one person to another. The infectious disease may be a zoonotic diseases, which is an infectious diseases of animals that can cause disease when transmitted to humans.

The infectious disease may be selected from the group consisting of Acute Flaccid Myelitis (AFM), Anaplasmosis, Anthrax, Babesiosis, Botulism, Brucellosis, Burkholderia mallei (Glanders), Burkholderia pseudomallei (Melioidosis), Campylobacteriosis (Campylobacter), Carbapenem-resistant Infection (CRE/CRPA), Chancroid, Chikungunya Virus Infection (Chikungunya), Chlamydia, Ciguatera, Clostridium difficile Infection, Clostridium perfringens (Epsilon Toxin), Coccidioidomycosis fungal infection (Valley fever), Creutzfeldt-Jacob Disease, transmissible spongiform encephalopathy (CJD), Cryptosporidiosis (Crypto), Cyclosporiasis, Dengue , 1,2,3,4 (Dengue Fever), Diphtheria, E. coli infection (E.Coli), Eastern Equine Encephalitis (EEE), Ebola, Hemorrhagic Fever (Ebola), Ehrlichiosis, Encephalitis , Arboviral or parainfectious, Enterovirus Infection , Non-Polio (Non-Polio Enterovirus), Enterovirus Infection , D68 (EV-D68), Giardiasis (Giardia), Gonococcal Infection (Gonorrhea), Granuloma inguinale, Haemophilus Influenza disease, Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster , zoster VZV (Shingles), Histoplasmosis infection (Histoplasmosis), Human Immunodeficiency Virus/AIDS (HIV/AIDS), Human Papillomarivus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LVG), Malaria, Measles, Meningitis, Viral Meningitis, Meningococcal Disease , Bacterial (Meningitis, bacterial), Coronavirus (CoV), COVID-19, Middle East Respiratory Syndrome Coronavirus (MERS-CoV), Mumps, Norovirus, Paralytic Shellfish Poisoning (Paralytic Shellfish Poisoning, Ciguatera), Pediculosis (Lice, Head and Body Lice), Pelvic Inflammatory Disease (PID), Pertussis (Whooping Cough), Plague; Bubonic, Septicemic, Pneumonic (Plague), Pneumococcal Disease (Pneumonia), Poliomyelitis (Polio), Powassan, Psittacosis, Pthiriasis (Crabs; Pubic Lice Infestation), Pustular Rash diseases (Small pox, monkeypox, cowpox), Q-Fever, Rabies, Ricin Poisoning, Rickettsiosis (Rocky Mountain Spotted Fever), Rubella , Including congenital (German Measles), Salmonellosis gastroenteritis (Salmonella), Scabies Infestation (Scabies), Scombroid, Severe Acute Respiratory Syndrome (SARS), Shigellosis gastroenteritis (Shigella), Smallpox, Staphyloccal Infection , Methicillin-resistant (MRSA), Staphylococcal Food Poisoning , Enterotoxin - B Poisoning (Staph Food Poisoning), Staphylococcal Infection , Urinary Tract Infection (UTI), Vancomycin Intermediate (VISA), Staphylococcal Infection , Vancomycin Resistant (VRSA), Streptococcal Disease , Group A (invasive) (Strep A), Streptococcal Disease , Group B (Strep-B), Streptococcal Toxic-Shock Syndrome , STSS, Toxic Shock (STSS, TSS), Syphilis , primary, secondary, early latent, late latent, congenital, Tetanus Infection, tetani (Lock Jaw), Trichonosis Infection (Trichinosis), Tuberculosis (TB), Tuberculosis (Latent) (LTBI), paratuberculosis (ParaTB), Tularemia (Rabbit fever), Typhoid Fever, Group D, Typhus, Vaginosis, bacterial (Yeast Infection), Varicella (Chickenpox), Vibrio cholerae (Cholera), Vibriosis (Vibrio), Viral Hemorrhagic Fever (Ebola, Lassa, Marburg), West Nile Virus, Yellow Fever, Yersenia (Yersinia) and Zika Virus Infection (Zika).

Nucleic acids that are biomarkers for infectious diseases are known.

The method may comprise diagnosing a coronavirus if the S Gene from the coronavirus is present. For example, the method may comprise diagnosing COVID-19 if the S Gene from 2019-nCoV is present. The method may comprise diagnosing SARS (severe acute respiratory syndrome) if the S Gene from SARS-CoV (2003) is present.

Preferred features for the second and subsequent aspects of the invention are as for the first aspect of the invention mutatis mutandis.

In one embodiment, the invention provides a microsensor for real time electrochemically or electrically monitoring and detecting nucleic acid (NA) amplification products, eg after each polymerase chain reaction cycle, utilizing electrochemically active or electrically conductive reporter materials. A variable power is applied to heat the reaction chamber with precise control of temperature. An electric voltage is applied, and electrical currents are recorded during a PCR amplification process to the electrodes that is suitable for quantifying the amplified products of a sample's nucleic acid(s) produced. This microsensor is suitable for point-of-use applications, e.g. detecting bioanalytes in remote locations.

The microsensor is fully integrated on porous silicon (PSi) and made of electroplated novel metals and thermoplastic layers through wet chemistry and thermal bonding and comprises an electrochemical transducer (three electrodes, electrochemical cell) covered with a reaction chamber at the top and a thermal block and transducer with two terminals at the bottom. The PSi on the bottom is connected to a variable power source and a control unit through the two terminals.

The temperature required for the biochemical reaction (nucleic acid amplification) is applied directly on the electrochemical transducer at the top and controlled by reading the changes in the resistance of the PSi at the bottom using the control unit. The electrochemical transducer at the top is connected to an electrochemical reader and provides a signal (electrochemical signal) related to species capable of exchange electrons.

A solution containing the NA sample, the reagents required for its amplification and the reporter are introduced in the reaction chamber on the electrochemical transducer. The reporter is an electroactive molecule which reacts selectively with the NA. The thermal block of the microsensor heats the sample within the reaction chamber to amplify the nucleic acid producing a solution containing the target amplicon which reacts with the reporter. The reporter free in solution reacts with the electrochemical transducer to produce an electrochemical signal that indicates the presence of the target amplicon. The current produced by the reaction of the reporter with the electrode, inversely proportional to the amount of amplicon, is recorded.

The inventors have developed a device providing a low-cost, simple, structured, portable PCR-EC platform based on the integration of conductive, hydrophobic and insulator layers on porous silicon (PSi). As a model disease, we are focussed on paratuberculosis (ParaTB) which primarily affects ruminant livestock such as cattle and sheep and is caused by the bacterial pathogen Mycobacterium avium subspecies paratuberculosis (MAP). The gold standard for the diagnosis of MAP is culturing the bacteria, which takes up to 12 weeks and is only possible in highly equipped laboratories. Direct detection of MAP antibody with ELISA has been extensively applied but the clinical sensitivity and specificity is lower than the molecular assays. Many real-time, conventional, semi-nested and nested PCR assays have been developed for the detection of MAP in up to 3 hours. Nevertheless, a big challenge is the implementation of PCR at the POC, because of its rapid thermocycling between the denaturation temperature, 95° C., and approximately 63° C. for primer annealing together with a precise temperature control. To overcome this problem, a custom board is connected to the PCR-EC platform. In this way, real-time PCR amplification and detection of DNA can be performed simultaneously with strict control of the temperature. The detection during the DNA amplification may be carried out free in solution using Methylene Blue (MB) as electrochemical reporter.

The invention may be used as follows:
1. A patient presents any symptom of a pathogenic disease or the control of a pathogenic disease is required.
2. Clinical personnel takes the sample from the patient and performs the sample treatment to extract the DNA through standard protocols.
3. The DNA is mixed with the provided reaction mixture and introduced in the device.
4. The real-time nucleic acid amplification is performed in the device within two hours.
5. The reading of the signal determinates the presence of the pathogen in the patient.

No changes are therefore needed in associated clinical practice. The device improves early diagnosis. The device can be used by clinical personnel at the point-of-care or need. It provides improved sensitivity, specificity, portability and ease-of-use.

Further advantages of the present invention include:

[1] The device utilises electrochemical detection which is cheaper than fluorescence and does not require a transparent nucleic acid amplification chamber, as required by fluorescent assays.

[2] Fluorescent molecules are susceptible to photobleaching hence require careful handing in the presence of light.

[3] The device exploits the temperature dependent semiconducting properties of silicon and controls the temperature through changes in the resistance of the substrate itself. This enables accurate control of the temperature of the sample, which is essential to the PCR and improves the accuracy of the process while shortening the time needed per test.

[4] The semiconductor substrate itself is used as the heater hence no additional heaters are needed to run the system.

[5] The device is manufactured through wet chemistry and thermal bonding, consequently negating the need of a cleanroom or time-consuming expensive equipment such as plasma deposition tools.

[6] The device is low-cost and portable in comparison to existing commercialised benchtop technologies.

The present invention will now be described by way of reference to the following Examples and accompanying Drawings which are present for the purposes of illustration only and are not to be construed as being limiting on the invention.

### EXAMPLE 1

### Fabrication and Setup

The fabrication of TriSilix chips (dimension of each chip: 1×1 cm) starts with a Si wafer (FIG. 1A). In this study we used lightly doped 4″ Si wafers (Siegert) however, wafers with a larger diameter would also work. To enable cleanroom-free fabrication, we have developed a series of methods to avoid photolithography and vacuum deposition/etching, limiting the fabrication process to wet etching/deposition, thermal bonding and laser cutting for patterning (FIG. 1B). First, each wafer is plated electrolessly for 20 s (in 80 μM KAuCl4 and 0.5% HF) to form a thin layer of gold particulate film and placed inside an etching bath containing an aqueous solution of H₂O₂ and HF with a ratio of 1:20 (30% H₂O₂: 10% HF v/v) to perform metal-assisted chemical etching (MACE) of Si for 10 min (i in FIG. 1B). All chemicals, unless otherwise stated, were purchased from Sigma.

This process forms a ˜200 nm thick porous Silicon (pSi) layer on each side of the wafer. Nanoporous pSi plays a critical role in the fabrication of TriSilix. The porous surface allows electroplating of high-quality metal films on the surface of the Si substrate by creating an interlocking, high porosity surface to improve adhesion. Without this step, the electroplated metal films do not adhere to the surface of the substrate. The pSi layer also allows thermal bonding of sheets of polymer films patterned by laser cutting in an ordinary heat press. After the formation of pSi surface, two layers of polyethylene terephthalate-ethylene (PET-ET, UK Insulations Ltd) are thermally bonded on the bottom and top surfaces of the Si wafer (ii in FIG. 1B) by heat pressing at 180° C. for 5 min (a Vevor HP230B heat press was used for the process). The heat pressed layers define the shape of the Working Electrode (WE) on the top surface and electrical contacts for Joule heating/resistance measurements on the bottom surface of the wafer. The polymer sheets patterned and heat pressed essentially act as masking layers for electroplating of the metal electrodes on the pSi surface equivalent to photolithography-based patterning in conventional microfabrication. Next, the unmasked areas of the nanoporous pSi surface were cleaned in 5% HF and electroplated by Au in a bath containing an aqueous solution of 10 mM KAu(CN)₂/KCN for 10 min under a constant current of 10 mA versus Pt wire (Alfa Aesar) yielding —100 nm thick porous Au films (iii in FIG. 1B). All chemicals, unless otherwise stated, were purchased from Sigma.

The electroactive area was evaluated by cyclic voltammetry adding 50 μL of 2 mM K₄[Fe(CN)₆] solution (0.1 M KlI) on the device sweeping the potential from −600 to +700 mV vs Ag at 100 mV s⁻¹. All reagents were purchased from Sigma. The dependence of the peak current on the scan rate was evaluated by cyclic voltammetry sweeping the potential from −400 to +600 mV vs Ag at 10, 50, 75, 100, 150 and 200 mV s⁻¹. The electrochemical cell of the device (Au-PSi Working electrode, Ag

Reference and Counter electrodes) was connected to the potentiostat (PalmSens3 model from PalmSens, UK) with flat crocodile clamps. Metallic wires were purchased in Alfa Aesar. According to the Randles-Sevcick equation for a flat electrode and for diffusion-controlled processes at 25° C. ip=(2.69·105)n^{3/2} A D^{1/2} C* v^{1/2}. Where ip is the peak current (A), n is the number of electrons transferred (n =1 for ferrocyanide), A the effective area of the electrode (cm²), D is the diffusion coefficient of ferrocyanide in aqueous solutions (6.50×10⁻⁶cm²s⁻¹), C* is the concentration (2×10⁻⁶ mol cm⁻³) and v is the scan rate (V s⁻¹). Cyclic voltammograms, as those shown in (FIG. 2) were recorded using five different devices without washings between scans. The gradient of the logarithmic plot peak current intensity vs the scan rate was 0.71±0.03 (R²=0.995) and 0.69±0.05 (R²=0.997), for anodic and cathodic processes, respectively. Readjusting the Randles-Sevcik equation with the experimental gradient values, calculated effective areas were 2.0±0.3 mm2 and 1.9±0.5 mm2 from anodic and cathodic data, respectively. Using the Randles-Sevcik equation with experimentally measured gradient values of 0.71±0.08, we determined that the Au film has a 2× larger electroactive area than the geometrically defined area due to the high porosity of the Au film.

To achieve high uniformity when electroplating across the wafer, we have designed, and 3D printed with polylactic acid (PLA) a custom holder which has a circular contact around the edges of the wafer (FIG. 3). The PSi wafer with round flexible Cu contacts is placed on the base (i in FIG. 3). Then, the reservoir of the holder (ii in FIG. 3) is placed on the top and the holder is closed through five screws. Since we observed a progressive leaking of the solution with the time, rubber 0-rings were integrated in both, base and reservoir, components and a third component (iii in FIG. 3) was incorporated in order to apply an inner pressure in the region of the O-rings.

To form a three-electrode electrochemical cell on the top surface, the Counter (CE) and Reference Electrodes (RE) created by heat pressing (iv in FIG. 1B) an Ag-plated Cu-PET-ET film patterned once again by laser cutting (using a fiber laser Speedy 100 fiber 20 W, Trotec). Ag plating was performed in an aqueous solution of 10 mM AgCN/KCN under an applied current of 20 mA versus Pt flat electrode (SPA plating). Once the basic device structure is finished, the wafer is laser-diced into individual chips (37 chips/4″ Si wafer) and a sample reservoir (polyethylene; diameter: 7 mm; volume: 20-40 μl) with two holes was thermally bonded across the three-electrode electrochemical cell at 110±10° C. (v in FIG. 1B). The sample reservoir is specifically designed to prevent the evaporation of the solvent during amplification of DNA at elevated temperatures. The final TriSilix chip has a thickness of 490 μm (N=7) in the WE region and 650 μm in the RE/CE region (N=7) without the sample reservoir. All chemicals, unless otherwise stated, were purchased from Sigma.

A schematic representation of the setup is shown in (FIG. 4). The electrochemical cell at the top is connected to an electrochemical reader (potentiostat, PalmSens 3) and provides a signal (electrochemical signal) related to species capable of exchange electrons with the working electrode (WE) or susceptible to redox reactions. The temperature required for the biochemical reaction (nucleic acid amplification) is applied directly on the electrochemical transducer at the top and controlled by reading the changes in the resistance of the PSi at the bottom using a control unit (custom board). The PSi at the bottom is connected to a variable power source and the custom board through the two gold contacts. To heat up the device, different currents are applied to the Au—PSi—Au diode (PSi with two Au contacts at the bottom) corresponding to the temperature required. Every 0.5 s a constant current of 10 mA is applied, and the voltage passed through the Au—PSi—Au is measured by the controller, the resistance is calculated by a custom software through the Ohm's law.

### EXAMPLE 2

### Characterization of Temperature Transduction

NA amplification reactions require maintaining the sample at a temperature setpoint with high precision. For PCR, the duration of each heating step must also be carefully controlled. Because the TriSilix uses Si, the Si substrate itself can be used both as an electrical heater and temperature sensor without adding any additional components for temperature transduction. Si, a semiconductor, heats up when an electrical current passes through it. Because Si has a high thermal conductivity (˜150 W/(m K) at 300° K), the substrate can be heated uniformly. The electrical resistance of Si is also dependent on temperature with a negative slope; the electrical resistance of Si drops with increasing temperature due to generation of mobile charge carriers allowing the use of Si substrate itself as a sensitive sensor of temperature.

We have applied electrical currents in the range of 0-400 mA between two Au electrodes deposited on the bottom of TriSilix chip to heat up the device electrically. During this experiment, we used a thermal camera (FLIR E4) to measure the temperature across the chip as a reference measurement. As illustrated in (FIG. 5A), TriSilix chip can be heated up to 100° C. with the application electrical currents up to 400 mA. The relationship between the current applied and substrate temperature measured was linear (slope: 232.3±8.7° C. A⁻¹; R²=0.9971).

By measuring the electrical resistance of the Si substrate using the two electrical contacts at the bottom of the chip, the temperature of the TriSilix chip can also be precisely identified which is important for correct execution of the amplification process (FIG. 5B). At room temperature, the electrical resistance of a TriSilix chips were 5.4±0.6Ω (N=37, corresponding to a batch) which varied linearly with temperature. Although we expected formation of Schottky barriers and non-linear electrical characteristics across the Au-Si interface, the I-V measurements (FIG. 5C) demonstrated that the junction exhibits relatively ohmic behavior with a linear I-V curve between −5V and +5V with a slope and regression coefficient (R²) of 0.056±0.006 A V⁻¹ and 0.9996±0.0003, respectively.

We have designed a custom electrical circuit (FIG. 5D) and MATLAB-based graphical user interface to provide programmable recipes for the thermocycling needed during NA amplification. The custom electrical circuit consisted of a voltage controlled constant current source and a differential amplifier. The degree of precision on the temperature control of the NA amplification chamber is related to the accuracy in establishing the resistance—temperature correlation curve for an Ohmic resistor: R=R₀(1+α(T−T₀)) where R and R0 are resistances of the thermistor at temperatures T and T₀ (reference temperature), and α is the temperature coefficient of resistance. This equation is used to convert the resistance reading (calculated from the voltage through the diode connected to the custom board, measured at a constant current of 10 mA every 0.5 s) to the temperature reading of the thermal camera. When R/R₀ is plotted against T-T₀, linear graphs (FIG. 5E) with a regression coefficient of 0.9991±0.0007 are obtained using 5 different devices. The slope of this linear graph gives an a value of 6.1±0.4×10-3° C⁻¹. With the well-calibrated temperature sensors, fast thermal cycling (heating and cooling gradients of 3.2 and 2.5° C.·s¹, respectively) of the NA amplification chamber (FIG. 5F) and temperature precision of ±1.3° C. are achieved when the values measured by the FTIR camera (black line) are compared with those calculated (grey line) from the recorded resistances (red line). The program of currents to heat the chamber during a PCR cycle was performed as follows(Figure 5F): i) denaturalization (94° C.): 410 mA for 23 s (1) and 400 mA for 30 s (2); ii) primer annealing (63° C.): 0 mA for 10 s (3) and 270 mA for 30 s (4); iii) extension (72° C.): 315 mA for 3 s (5) and 310 mA for 30 s (6); electroanalysis (40° C.): 0 mA for 12 s (7) and 170 mA for 30 s(8).

### EXAMPLE 3

### Electrochemical Characterization of the Redox Reporter

Electrochemical detection, electrical heating and thermoelectric sensing were simultaneously performed by connecting the device using a thermostable interface which consists of a 3D-printed PLA case filled with dragon skin (soft and stable material under NA amplification conditions). There are five flat stainless-steel connectors embedded in the dragon skin, three at the top to connect WE, RE and CE electrodes to the potentiostat and four at the bottom to connect the Au—PSi—Au diode to the custom board. The electrochemical approach we use for the detection of DNA involves the use of methylene blue (MB), a redox-active reporter that is intercalated between guanine-cytosine base pairs of the double-strand DNA (ds-DNA). Intercalated molecules MB into the ds-DNA reduce the concentration of free MB in solution, leading to a decreased redox signal during electroanalysis.

First, we characterized the redox processes of MB on the developed device by cyclic voltammetry (CV) at room temperature in a 125 μg mL⁻¹ MB solution in 10 mM phosphate-buffered saline (PBS) pH 7 sweeping the potential from −400 to 200 mV at 100 mV s⁻¹. The results using five devices indicate (FIG. 6A) anodic and cathodic process at −0.67±2 and −0.97±4 mV versus Ag, respectively. We chose a potential window from −0.5 to −0.25 V versus Ag, (corresponding to the anodic process) to perform square wave voltammetry (SWV), electroanalytical method more sensitive for the rest of experiments. FIG. 6B illustrates recorded SWVs in MB solutions and phosphate-buffered saline (PBS) with a range of concentrations from 0 to 150 μg mL⁻¹. The calibration plot, peak current intensity versus MB concentration (inset), from SWVs from 5 different devices, shows a dynamic linear range from 0.5 μg mL⁻¹ to 80 μg mL⁻¹ (R²=0.9963). All chemicals, unless otherwise stated, were purchased from Sigma. A PalmSens 3 potentiostat was used to perform electrochemical measurements.

It is important to know the electrochemical behavior of the reporter (MB) under the amplification temperature. Once connected all the components (circuit board, power supply and potentiostat), SWVs were recorded in 30 μL MB solutions with a range of concentrations from 0 to 50 μg mL⁻¹ in PBS at 40° C. after joule heating at 40 (RPA) and 94 (PCR) ° C. for 5 min. 10 μL of mineral oil were added to the solution to prevent any evaporation. The resulting peak current intensity versus MB concentration plots (from SWVs recorded using 5 different devices) in FIG. 6C reveal a clear effect of the temperature on the electroanalytical performance. The sensitivity is ˜2.3 and ˜3.5 times greater than at room temperature after heating at 40 and 94° C., respectively, probably due to a preconcentration of MB on the WE. On the other hand, the irreproducibility of this thermal effect leads to the loss of precision with the temperature, thus, to a higher limit of detection. These limitations must be considered to choose the concentration of the reporter in NA amplification-based analysis. We chose 10, 20 and 30 μg mL⁻¹ MB concentrations for real-time RPA (RT-RPA), Mycobacterium avium paratuberculosis PCR (MAP RT-PCR) and Coronavirus PCR (CoV RT-PCR) amplification experiments, respectively.

### EXAMPLE 4

### NA Amplification

The positive control DNA (3.2 kbp template, 144-bp amplicons) from the TwistAmp Basic kit (TwistDx, UK) and 100-bp CTX-M ESBL (extended-spectrum beta-lactamases, 5′-ATTGACGTGC TTTTCCGCAA TCGGATTATA GTTAACAAGG TCAGATTTTT TGATCTCAAC TCGCTGATTT AACAGATTCG GTTCGCTTTC ACTTTTCTTC-3′) DNA as negative control were used in a first set of experiments to study the electroanalytical signal and thermal stability of the platform with the time under isothermal amplification conditions. Unlike PCR, isothermal DNA amplification assays do not need a controlled thermal cycling. RPA is an isothermal technology, which amplifies DNA at a constant temperature between 25° C. and 42° C. As specified by the manufacturer, 50 μL of Mastermix (every component but OAc and DNA) and RPA mix solutions were freshly prepared. The original TwistDx assay was slightly modified to include 10 μg mL⁻¹ of MB. First the RPA pre-mix was prepared by mixing in a vortex 25 μL of 2× buffer, 8 μL of 10 mM dNTPs (Fisher Scientific), 5 μL of 10X E Mix and 4 μL of control primer Mix (30 bp). 2.5 μL of 20× core solution and 1 μL of 0.05% MB (Sigma) were placed in the lid and mixed with 10 inversions. All chemicals, unless otherwise stated, are included in the TwistAmp Basic kit (TwixDx). Then, the mastermix (every reagent but MgOAc salt and DNA) was prepared by adding 3.5 μL of Nuclease-free ultrapure water (Fisher Scientific) to the pre-mix solution followed by vortex. 30 μL of this solution were added into the device chamber followed by 10 μL of mineral oil (Sigma).

SWVs were recorded every 2.5 min of amplification at 40° C., sweeping the potential from −400 to +600 mV at 50 Hz with 100 mV of pulse amplitude. The average signal from SWVs recorded every 2.5 min in Mastermix solutions at 40° C. and room temperature (FIG. 7A) show good stability and intensity over the time for 40 min.

Electrochemical real-time RPA (RT-RPA) was performed in five positive and negative DNA controls (RPA mix solutions). With this aim RPA mixes were prepared by adding, instead of nuclease-free water, 2.5 μL of 280 mM MgOAc and 1 μL of positive control DNA or 50 nM negative control (CTX-M ESBL) solution to the pre-mix solution. All chemicals, unless otherwise stated, were included in the TwistAmp Basic kit. The EC signal depends on the amount of MB and then the higher the amount of ds-DNA the lower the MB signal. The RT-RPA plot is the graphical representation, versus RPA time, of the peak current intensities from SWVs recorded at time t normalized respect to those at time 0. This plot (FIG. 7B) shows evidence of DNA amplification of the positive control within the first 10 minutes of RPA. The negative samples show no or slightly indication of DNA amplification.

RPA assaysoffer greater utility for POC NA analysis by including simplistic reactor designs or portable heat sources. However, RPA only allows the amplification of 100-200-bp target sequences and is less specific due, mainly, to the initiation of the reaction at relative low temperatures. Besides, the high-precision thermal sensing of the developed device can be exploited for RT-PCR analysis.

MAP K10 strain (caw) solution was acquired from Moredun Research Institute (Edinburgh, Scotland EH26 OPZ, UK). A 40 pg μL⁻¹ concentration of DNA template was confirmed by gel electrophoresis. The forward primer, 5′-GCC GCG CTG CTG GAG TTG A-3′ (Biomers), and reverse primer, 5′- CGC GGC ACG GCT CTT GTT -3′ (Biomers), were used to amplify 563 nucleotides of the IS900 gene of M. paratuberculosis (204-766 of GenBank accession number AE016958.1; National Center for Biotechnology Information, USA). We performed a titration experiment at room temperature by SWV in 20 μg mL⁻¹ MB MAP PCR mix solutions in order to know the range of concentrations of DNA to be amplified. First, a MAP PCR pre-mix was prepared by mixing 40 μL of 1.5 M Tris-HCl Buffer pH 8.8 (Bio-Rad), 20 μL of 10 mM dNTPs, 20 μL of 10 pmol μL⁻¹ forward primer (Biomers), 20 μL of 10 pmol μL⁻¹ reverse primer (Biomers), 20 μL of 0.5 U μL⁻¹Taq DNA polymerase and 640 μL of Nuclease-free ultrapure water and 40 μL 0.05% MB were added in a 1 mL eppendorf tube and mixed in vortex. All chemicals, unless otherwise stated, were purchased from Fisher Scientific. Then PCR Mix was prepared by adding 4 μL of 25 mM MgCl2, 5 μL of Nuclease-free ultrapure water (Fisher Scientific) and 1 μL of genomic DNA aqueous solution (dilution of the 40 pg μL-1 K10 MAP strain Nuclease-free ultrapure water from Moredun Institute) were added to 40 μL of MAP PCR pre-mix and mixed in vortex. 50 μL of this solution were added into the device chamber. We swept the potential from −400 to +600 mV (anodic process) and from −600 mV to 0 mV (cathodic process) at 50 Hz with 100 mV of pulse amplitude. The 50 μL PCR mix contained 60 mM Tris-HCl buffer (pH 8.8), 2.0 mM MgCl2, 0.2 mM of each of the four dNTPs, 10 pmol of each of the primers, 0.5 U Taq DNA polymerase, 2 μL of 0.05% MB aqueous solution and 5 μL of DNA. FIG. 7C shows the anodic and cathodic tritration curves (peak current intensities versus genomic DNA concentration) in a 2-200 pg range at room temperature. The peak current intensity decreased linearly with the concentration of DNA up to 40 pg. A plateau is reached for higher DNA concentrations suggesting that all the MB molecules have reacted. Although previous experiments were based on the anodic process of MB, the sensitivity (−2.53 μA pg-1) is higher for the cathodic process in comparison with that obtained from anodic SWVs (sensitivity of −1.46 μA pg⁻¹). In addition, the LOD of non-amplified DNA, calculated as 3Sb/b (where b is the slope and Sb its standard deviation of the blank) was lower for the cathodic process (0.8 pg) than that for the anodic process (1.2 pg). Therefore, we chose a potential window from −400 to +600 mV corresponding to the cathodic process of MB, and a range of concentrations from 10 to 500 fg of DNA (below the LOD) to perform MAP RT-PCR. Electrochemical real-time PCR (RT-PCR) was performed in five Mastermix solutions and five MAP PCR mix solutions in a range of concentrations from 10 to 500 fg of MAP K10 strain. To prepare mastermix solutions (every reagent except DNA and MgCl₂) 10 μL of Nuclease-free ultrapure water (Fisher Scientific) were added to 40 μL of MAP PCR pre-mix and mixed in vortex. MAP PCR mix solutions were prepared as previously described. 30 μL of MAP PCR mastermix or MAP PRC mix were placed in the device followed by 10 μL of mineral oil. SWVs were recorded every 5 cycles with a PCR temperature profile as follows: initial activation at 94° C. for 1 min, 5 cycles of 94° C. for 30 sec, annealing at 63° C. for 30 sec, extension 72° C. for 30 sec, and SWV recording at 40° C. for 30 s. The RT-PCR curves, normalized peak current intensity versus PCR cycle (FIG. 7D), shows evidence of DNA amplification for concentrations higher than 50 fg while it is unclear that 20 fg and the blank (water) could provide different responses. The limit of detection of 50 fg by employing the genomic DNA, corresponding to approximately 10 MAP genomes as one MAP genome has a weight of 5.29 fg. The results were corroborated by analyzing the same K10 genome sample using the IDVet commercial qPCR kit and equipment (fluorescence detection) where the cut-off for positives is Ct 40 (samples with Ct>33 are re-tested as standard to confirm the original positive result). Ct of 35 and 32 (FIG. 7E) were obtained for 20 fg and 40 fg respectively, therefore, TriSilix compares very well with the commercial qPCR.

Finally, we performed CoV RT-PCR experiments using synthetic cDNA fragments (IdtDNA). As positive sample we added a solution of synthetic cDNA fragment corresponding to 22712-22869 nucleotides of SARS-CoV.2 (GenBank accession number MN908947), the one responsible of the current COVID-19 disease from Wuhan Market. As negative sample, we added a solution of synthetic cDNA fragment corresponding to 17741-17984 nucleotides of SARS-CoV (AY274119), the one responsible of the Middle East disease in 2003. The forward primer, 5′- CCT ACT AAA TTA AAT GAT CTC TGC TTT ACT-3″ (Biomers), and reverse primer, 5′- CAA GCT ATA ACG CAG CCT GTA -3″ (Biomers), were used to amplify the 158 nucleotides of cDNA from SARS-COV.2. First, a CoV PCR mastermix was prepared by mixing 25 μL of 10 pM of each primer (Biomers), 5 μL of 0.5 U μL⁻¹ of Taq Polymerase (Fisher Scientific), 5 μL of 10 mM dNTPs, 25 μL of 10X Taq Polymerase buffer (‘+KCl—MgCl2’) provided with the enzyme (Fisher Scientific) and 115 μL of Nuclease-free water (Fisher Scientific). Then the CoV PCR mix was prepared by adding 3 μL of 0.005% Methylene Blue (MB)(Sigma Aldrich), 1 μL of 1 pg μL⁻¹ SARS-COV.2 or SARS-COV cDNA solution, 4 μL of 25 mM MgCl2 (provided by the Taq Polymerase kit, Fisher Scientific) and 2 μL of nuclease-free water (Fisher Scientific) were added to 40 μL of CoV PCR mastermix. At the end, the COV PCR mix consists of 0.5 U Taq polymerase, 1X Taq Polymerase buffer, 1 μM forward primer, 1 μM reverse primer, 2 mM MgCl2, 0.2 mM dNTPs, 30 pg mL⁻¹ MB and 1 pg cDNA (positive or negative). 30 μL of positive or negative CoV PCR mix solutions were placed in the device followed by 10 μL of mineral oil.

SWVs were recorded every 5 cycles with a PCR temperature profile as follows: initial activation at 94° C. for 1 min, 5 cycles of 94° C. for 30 sec, annealing at 63° C. for 30 sec, extension 72° C. for 30 sec, and SWV recording at 40° C. for 30 s. The RT-PCR curves, normalized peak current intensity versus PCR cycle (FIG. 8), shows evidence of DNA amplification for the positive sample different from the negative sample at the 35^{th} cycle. Therefore, using this device and the described method we are able to detect cDNA from SARS-COV.2 and distinguish between strands from different SARS-COV viruses.

1. A device for detecting a substance in a solution, wherein the device comprises:
a substrate comprising silicon;
a first electrode for use as a working electrode in an electrochemical cell and coupled to the substrate; and
Ohmic contacts coupled to the substrate and configured to pass a current through the substrate when connected to a power source.

2. The device of claim 1 wherein the substrate comprises one or more bulk silicon layers, and optionally one or more porous silicon layers and/or one or more silicon dioxide layers.

3. The device of any preceding claim, wherein the substrate comprises one or more porous silicon layers, optionally wherein the one or more porous silicon layers have around 30 nm to around 60 nm wide pores and/or the depth of the pores is around 600 nm.

4. The device of any preceding claim, wherein first electrode comprises a noble metal or carbon, optionally wherein the first electrode is a gold electrode.

5. The device of any preceding claim, further comprising a second electrode for use as a counter electrode in the electrochemical cell, wherein the second electrode is coupled to the silicon comprising substrate via a first insulating layer.

6. The device of any preceding claim, further comprising a third electrode for use as a reference electrode in the electrochemical cell, wherein the third electrode is coupled to the silicon comprising substrate via a second insulating layer.

7. The device of any preceding claim, wherein the first electrode is coupled to a first surface of the substrate, wherein the first surface is a porous silicon surface.

8. The device of any preceding claim, further comprising Ohmic contacts coupled to a second surface of the substrate, optionally wherein the second surface is a porous silicon surface.

9. The device of claim 8, wherein the Ohmic contacts comprise are
(a) gold Ohmic contacts, or
(b) aluminium and copper Ohmic contacts.

10. The device of any one of claim 8 or 9, wherein the Ohmic contacts and the first electrode are on opposite sides of the substrate, optionally wherein the first electrode is positioned between the Ohmic contacts.

11. The device of any one of claims 8 to 10, wherein the first electrode is electrically insulated from the Ohmic contacts.

12. The device of claim 11, wherein the substrate comprises electrically insulating layer, optionally wherein the electrically insulating layer comprises silicon dioxide.

13. The device of any preceding claim, wherein the first electrode is in electrical and thermal contact with the substrate.

14. The device of any preceding claim, further comprising a chamber for retaining the solution, wherein the first electrode is positioned within the chamber and optionally wherein the Ohmic contacts are positioned outside the chamber.

15. A system for detecting a substance in a solution, the system comprising:
a device according to any preceding claim; and
one or more of:
a potentiostat configured to control an electrochemical potential of the first electrode;
a power source configured to pass a current through the substrate via the Ohmic contacts; and
a control unit connected to the Ohmic contacts and configured to calculate a resistance of the substrate.

16. A method of fabricating a device according to any one of claims 1 to 14, the method comprising
electroplating the first electrode to the substrate, thereby coupling the first electrode to the substrate, and
electroplating the Ohmic contacts to the substrate thereby coupling the Ohmic contacts to the substrate.

17. The method of claim 16, wherein the first electrode and the Ohmic contacts may be simultaneously coupled to the substrate by electroplating.

18. The method of any one of claim 16 or 17, further comprising forming one or more porous silicon layers on the substrate prior to the electroplating, optionally wherein the one or more porous silicon layers are formed by Metal-assisted chemical etching (MACE), anodization, galvanization, photoetching, HNO₃/HF vapor etching, by mechanical means or by stain-etching.

19. The method of claim 18, wherein the porous silicon layer or layers is be formed by Metal-assisted chemical etching (MACE), optionally wherein the MACE uses a catalyst selected from the group consisting of Gold, Platinum, Silver, Nickel, Manganese, Cobalt, Copper, Chromium, Magnesium and Iron.

20. A method of detecting a substance in a solution using a device according to any one of claims 1 to 14, the method comprising
placing the solution in contact with the first electrode,
passing a current through the substrate, and
detecting a substance in the solution.

21. A method of diagnosis using a device according to the any one of claims 1 to 14, the method comprising
placing a sample in contact with the first electrode,
passing a current through the substrate, and
detecting a substance in the sample.

22. The method of claim 21, wherein the sample is an environmental sample or a clinical sample, optionally wherein the clinical sample is from a patient.

23. The method of any one of claim 21 or 22, wherein the sample has or is suspected to contain a pathogen, wherein the pathogen is a Coronavirus or Tuberculosis, optionally wherein the Coronavirus is SARS-CoV-2.

24. The method of any one of claims 20 to 23, comprising passing a current of around 150 mA to around 450 mA through the substrate.

25. The method of any one of claims 20 to 24, wherein the passing a current through the substrate is passing a sequence of currents through the substrate, wherein the sequence of currents comprises
(i) a first current of around 410 mA for around 23 seconds and a second current of around 380 mA to around 420 mA for around 15 to around 60 seconds;
(ii) an optional third current of around 0 mA for around 10 seconds and a fourth current of around 250 mA to around 290 mA for around 15 to around 60 seconds;
(iii) a fifth current of around 315 mA for around 3 seconds and a sixth current of around 290 to around 330 mA for around 15 to around 60 seconds;
(iv) a seventh current of around 0 mA for around 10 to 15 seconds and an optional eighth current of less than or equal to around 190 mA for around 30 seconds.


SEQUENCE LISTING (5 sequences)

SEQ ID NO: 1 - DNA, 100 bp, Artificial sequence
  100-bp CTX-M ESBL (extended-spectrum beta- lactamases)
attgacgtgcttttccgcaatcggattatagttaacaaggtcagattttttgatctcaactcgctgatttaacagattcggttcgctttcacttttcttc

SEQ ID NO: 2 - DNA, 19 bp, Artificial sequence
  Forward primer to amplify 563 nucleotides of the IS900 gene of M. paratuberculosis
gccgcgctgctggagttga

SEQ ID NO: 3 - DNA, 18 bp, Artificial sequence
  Reverse primer to amplify 563 nucleotides of the IS900 gene of M. paratuberculosis
cgcggcacggctcttgtt

SEQ ID NO: 4 - DNA, 30 bp, Artificial sequence
  Forward primer to amplify the 158 nucleotides of cDNA from SARS-COV.2
cctactaaattaaatgatctctgctttact

SEQ ID NO: 5 - DNA, 21 bp, Artificial sequence
  Reverse primer to amplify the 158 nucleotides of cDNA from SARS-COV.2
caagctataacgcagcctgta